(19) United States
(12) Patent Application Publication
(10) Pub. No.: US 2005/0059645 A1
(43) Pub. Date: Mar. 17, 2005

(54) Methods for the treatment of male and female sexual dysfunction

(76) Inventor: Nicholas S. Bodor, Gainesville, FL (US)

Correspondence Address: IVAX CORPORATION, 4400 Biscayne Boulevard, Miami, FL, 33137, US

(21) Appl. No.: 10/910,964

(22) Filed: Aug. 2, 2004

Related U.S. Application Data
(60) Provisional application No. 60/491,233, filed on Jul. 31, 2003.
(60) Provisional application No. 60/491,234, filed on Jul. 31, 2003.
(60) Provisional application No. 60/586,506, filed on Jul. 9, 2004.

(51) Int. Cl. A61K 31/58

(52) U.S. Cl. 514/176

(57) ABSTRACT

Methods for the treatment of female sexual dysfunction, including treatment of associated postmenopausal symptoms, are provided using very low doses of 17β-[(1-methyl-1,4-dihydro-3-pyridinyl)carbonyloxy]estra-1,3,5(10)-trien-3-ol, also known as E₂-CDS, which do not elevate average steady-state peripheral estradiol levels to above about 50-60 pg/ml. Also, methods for the treatment of male sexual dysfunction are provided using very low doses of E₂-CDS which do not substantially elevate average peripheral estradiol levels to above average normal peripheral levels in the male mammal.

### CROSS-REFERENCE TO RELATED APPLICATIONS

This application claims the priority of U.S. Provisional Applications No. 60/491,234, filed Jul. 31, 2003, No. 60/491,233, filed Jul. 31, 2003 and No. 60/586,506, filed Jul. 9, 2004 (Attorney Docket No. IVAX0024-P-USA, entitled “TRANSMUCOSAL DOSAGE FORMS FOR BRAIN-TARGETED STEROID CHEMICAL DELIVERY SYSTEMS”), each of which is incorporated by reference herein in its entirety and relied upon.

### BACKGROUND OF THE INVENTION

1. Field of the Invention

The invention relates to methods for the treatment of female and male sexual dysfunction using a brain-targeted delivery system for estradiol, namely 17β-[(1-methyl-1,4-dihydro-3-pyridinyl)carbonyloxy]estra-1,3,5(10)-trien-3-ol, also known as E₂-CDS.

2. Background of the Prior Art

Masters and Johnson defined sexual dysfunction as “the persistent impairment of normal or usual patterns of sexual interest and/or response” (Masters et al., Human Sexual Response, Boston, Mass.: Little, Brown and Co. 1966). The problem came to national attention when the results of the National Health and Societal Life Survey were published in 1999. Interviews with over 3000 American men and women aged 18-59 revealed that 31% of men and 43% of women (about 40 million) experienced some degree of sexual dysfunction. The scope of the problem was such that it was said to “warrant recognition as a significant public health concern.” See Laumann et al., “Sexual Dysfunction in the United States: prevalence and predictors,” JAMA 281:537 (1999). Although sexual dysfunction rarely threatens physical health, it can take a heavy psychological toll, bringing on depression, anxiety, and debilitating feelings of inadequacy.

Sexual dysfunction in men includes, in the main, erectile dysfunction, male orgasmic disorder, inhibited or hypoactive sexual desire and priapism. Inhibited or hypoactive sexual desire refers to a decrease in desire for, or interest in, sexual activity and can result from a variety of causes, including physical illness, depression, hormonal abnormality or medications that affect libido.

Male sexual behavior is composed of proceptive and consummatory behaviors. The proceptive behaviors include the awareness of the presence of a receptive female, the pursuit of that female and the positioning of the body (mounting) to allow insertion of the penis into the vagina. This latter behavior, termed intromission, as well as its prerequisite erection of the penis and eventual ejaculation, are the consummatory components of masculine sexual behavior. The accomplishment of ejaculation requires the entire repertoire of the aforementioned behaviors. It is dependent upon the close coordination of sensory and motor components of the nervous system which are coordinated at the levels of the brain and by spinal reflexes.

Male sexual behavior is steroid-dependent and in most mammals testosterone, released by the Leydig cells of the testes, is the hormone involved in permitting the expression of masculine behavior. In sexually experienced male rats, castration results in a gradual diminution and an eventual extinction of masculine sexual behavior. Replacement of the hormone testosterone completely prevents the loss of masculine sexual behavior [Malmnas, Acta Physiologica Scand. (Suppl. 395): 9-46, 1973; Damassa et al., Hormones and Behavior 8: 275-286, 1977]. Additionally, if after castration masculine sexual behavior is allowed to wane prior to the initiation of testosterone replacement, testosterone replacement can restore the full expression of the behavior [Davidson et al., in S. Levine (Ed.) Hormones and Behavior, Academic Press, New York, 1972, pp. 63-103].

Several lines of evidence indicate that the proceptive components of masculine sexual behavior are dependent upon the aromatization in the brain of testosterone to estradiol. First, it has now been conclusively demonstrated that the blockade of the conversion of testosterone to estradiol with aromatase inhibitors or the blockade of estrogen actions with antiestrogens antagonize the effects of testosterone on masculine sexual behavior. Consistent with this notion of testosterone metabolism into estradiol which activates masculine sexual behavior is the distribution of the enzyme, aromatase. In mammals, aromatase is reported to be concentrated in the preoptic area, the hypothalamus, and the amygdala brain regions known to mediate the effects of testosterone on masculine sexual behavior. For representative literature, see: N. J. MacLusky, A. Philip, C. Hurlburt and F. Naftolin, in Metabolism of Hormonal Steroids in the Neuroendocrine Structures, Eds. F. Celotti, F. Naftolin and L. Martini, Raven Press, Vol. 13, 1984, pp. 103-116; B. S. McEwen, Science 211: 1303-1311, 1981; Beyer et al., Hormones and Behavior 7: 353-363, 1976.

Second, following the systemic administration of testosterone, estradiol appears as the major androgen metabolite in regions of the brain known to mediate masculine sexual behavior. In one study of Rhesus monkeys, [³H]-testosterone was administered systemically and sections of the brain were extracted to determine the steroid metabolites present. In the hypothalamus, preoptic area and amygdala, 50% or more of the radioactivity was reported to be estradiol, while in other brain structures, the radioactivity remained as testosterone. In contrast, in peripheral tissues such as the seminal vesicles, glans penis and the prostate gland, dihydrotestosterone was the major metabolite (R. W. Bonsall et al., Life Sciences 33: 655-663, 1983). In a subsequent study, which more extensively evaluated testosterone metabolism in brain regions, only the hypothalamus, preoptic area and amygdala were found to form estradiol significantly (61, 43 and 64%, respectively). All other brain areas evaluated contained either no estradiol or less than 10% of the recovered steroids (R. P. Michael et al., Endocrinology 118: 1935-1944, 1986). Finally, the amount of estradiol bound to nuclear receptors in the preoptic area-hypothalamus is directly related to the level of testosterone in the serum, suggesting that the source of estradiol bound to its receptor in this brain region is circulating testosterone (L. C. Krey et al., Brain Res. 193: 277-283, 1980).

Third, estradiol stimulates the proceptive components of masculine sexual behavior. Pfaff(J. Comp. Physiol. Psych. 73: 349-358, 1970) administered estradiol benzoate systemically (10 μg/day) for 9 to 11 days to castrated male rats and observed that estradiol increased mounting, intromissions and ano-genital sniffing and reduced mounting latency to levels comparable to that observed following the administration of testosterone propionate (200 μg/day). Sodersten (Hormones and Behavior 4: 247-256, 1973) administered estradiol benzoate (100 μg/day) for 24 to 28 days to male rats castrated 6 weeks previously and found that mounts and intromissions were equivalent to those observed following similar treatment with testosterone propionate (100 μg/day). Ejaculations were less affected by estradiol benzoate then by testosterone propionate. Gray et al. (Physiology and Behavior 24: 463-468, 1980) administered Silastic pellets containing estradiol and evaluated sexual behavior 7 days later. They observed that estradiol stimulated mounting behavior but was less effective than testosterone in enhancing intromissions and ejaculations.

For the most part, estrogens have not been proposed to treat male sexual dysfunction, primarily because of significant undesirable side-effects. Estrogens are, however, administered to men in the treatment of prostatic carcinoma. Unfortunately, some significant toxic effects occur in the male: estrogen treatment stimulates gynecomastia, causes testicular regression and feminizes hair growth patterns in men.

In contrast to the widespread interest in research and treatment of male sexual dysfunction (MSD), less attention has been paid to the sexual problems of women. Few studies have investigated the psychological and physiological underpinnings of female sexual dysfunction (FSD) and fewer treatments are available for women than for men. A major barrier to the development of clinical research and practice has been the absence of a well defined, broadly accepted diagnostic framework and classification for female sexual dysfunction. In 2000, the Sexual Function Health Council of the American Foundation for Urologic Disease proposed such a classification system and it has been widely accepted and will be used herein; see Basson et al., J. Urol. 163:888 (2000).

The normal female sexual response cycle is now divided into four stages: desire, excitement, orgasm, and resolution. Female sexual dysfunction may occur at any one, or more than one, stage of the female sexual response cycle. Shepherd, J. Amer. Pharm. Assoc. 42(3):479(2002). Consensus classifications and definitions, based on the stages of normal female sexual response are divided into four broad categories: sexual desire disorders, sexual arousal disorders, orgasmic disorders, and sexual pain disorders; of these four, the most common is hypoactive (inhibited) sexual desire disorder (HSDD). HSDD is defined as persistent or recurrent deficiency (or absence) of sexual fantasies, thoughts, and/or desire for, or receptivity to, sexual activity, which causes personal distress. Researchers have reported that as many as 55% of all women presenting with complaints of FSD identity HSDD as the main reason; Wincze et al., Sexual Dysfunction, New York, N.Y.: Guilford Press; 1991. HSDD can result from, among other etiologies, physical illness, hormonal abnormality, or medications that affect libido.

Estrogens are among the most ubiquitous and important hormones in the female organism. Estradiol (E₂) is the most potent natural estrogen, as it has the highest affinity for estrogen receptors; Ruggiero and Likis, J. Midiwifery and Women's Health, 47(3), 103-138 (2002). Many of estradiol's pharmacological effects are mediated through the CNS. Brain-specific steroid deprivation causes syndromes such as male or female sexual dysfunction and menopausal vasomotor symptoms (“hot flushes”); see Greendale et al., Lancet, 353, 571-580 (1999); Yen, J. Reprod. Med. 18(6), 287-296 (1977). In postmenopausal women, sexual dysfunction may be closely linked to and include symptoms associated with the estrogen deprivation of menopause, such as vaginal dryness/lack of lubrication and consequent pain associated with intercourse, which can be closely associated in turn with diminished sexual desire. Other postmenopausal symptoms such as night sweats, hot flushes, insomnia, depression, nervousness, urinary incontinence, irritability and anxiety are also likely to be associated with diminished sexual desire. Returning estrogen levels to pre-menopausal levels to relieve the symptoms of menopause mentioned above as well as other menopausal symptoms such as osteoporosis has been the goal of HRT (hormone replacement therapy).

A brain-targeted chemical delivery system (CDS) represents a rational drug design approach which exploits sequential metabolism, not only to deliver but also to target drugs to their site of action. A dihydropyridine [symbol] pyridinium salt-type redox system has been previously proposed and applied to a number of drugs, including estradiol. According to this redox system, a centrally acting drug [D] is coupled to a quaternary carrier [QC]⁺ through a reactive functional group (such as a hydroxyl function) in the drug; the [D-QC]⁺ which results is then reduced chemically to the lipoidal dihydro form [D-DHC]. After administration of [D-DHC] in vivo, it is rapidly distributed throughout the body, including the brain. The dihydro form [D-DHC] is then in situ oxidized (by the NAD [symbol] NADH system) to the ideally inactive original [D-QC]⁺ quaternary salt which, because of its ionic, hydrophilic character, is rapidly eliminated from the general circulation of the body, while the blood-brain barrier prevents its elimination from the brain. Enzymatic change of the [D-QC]⁺ which is “locked” in the brain effects a sustained delivery of the drug species [D], followed by its normal elimination. A properly selected carrier [QC]⁺ will also be rapidly eliminated from the brain. Because of the facile elimination of [D-QC]⁺ from the general circulation, only minor amounts of the drug [D] will be released in the brain. The overall result will be a brain-specific sustained release of the target drug species. See, for example, Bodor U.S. Pat. Nos. 4,540,564; 4,900,837; 4,983,586; 5,002,935; 5,017,566; and 5,024,998; Bodor et al. U.S. Pat. No. 4,617,298; and Anderson et al. U.S. Pat. No. 4,863,911. The compound 17β-[(1-methyl-1,4-dihydro-3-pyridinyl)carbonyloxy]estra-1,3,5(10)-trien-3-ol, which has the structure 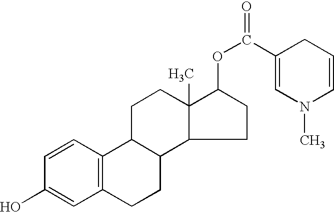
and is also known as E₂-CDS, is a specific CDS devised for estradiol which is described in these patents. In this case, the lipophilic 17-dihydrotrigonelline ester of estradiol, i.e., E₂-CDS, is enzymatically converted to the hydrophilic trigonellinate ester (E₂-Q⁺), which is specifically retained in the brain due to the characteristics of the BBB. The hydrophilic E₂-Q⁺ form is thus “locked-in” the brain and is slowly and sustainedly hydrolyzed by esterases to estradiol (E₂). Similar E₂-CDS→E₂-Q⁺ conversion in the rest of the body accelerates peripheral elimination and improves targeting.

E₂-CDS has been previously suggested for a number of uses, including treatment of male sexual dysfunction (Anderson et al U.S. Pat. No. 4,863,911) and weight control (Bodor et al. U.S. Pat. No. 4,617,298), as well as brain-specific, steroid deprivation syndromes (such as hot flushes) and for chronic reduction of gonadotropin secretion for fertility regulation (contraception) or treatment of gonadal steroid-dependent diseases, such as endometriosis and prostatic hypertrophy (noted in column 46 of Bodor et al. U.S. Pat. No. 4,617,298). These prior investigations focused on the ability of E₂-CDS to produce extremely long-acting effects (of the order of 1 month) from a single selected dose of E₂-CDS. This was considered highly desirable for dosing purposes, as once-a-month administration was deemed particularly convenient, especially so in women for purposes of contraception. Initially high levels of peripheral estrogen were not a concern, as they were lower than the levels produced by equimolar conventional estrogen, and LH reduction was comparable to that obtained with equimolar conventional estrogen. In rats, 3 mg/kg of E₂-CDS was typically used to provide activity for a period of 1 month. Such an amount is generally 10 times the mg/kg amount expected to be comparable in humans. Thus, a 0.3 mg/kg amount was expected to provide comparable results in women.

For an overview of prior work with E₂-CDS, see Brewster et al., Rev. Neurosci. 2, 241-285 (1990); also see Brewster et al, J. Pharm. Sci. 77:981-985 (1988), in which doses of E₂-CDS in 20% (w/v) HPPCD as low as 0.1 mg/kg i.v. gave LH suppression for as long as 18 days (but not 25 days) in castrated female rats.

Estes et al, Life Sciences 40, 1327-1334 (1987), described a series of four studies, two in orchidectomized rats and two (Experiments 3 and 4) in ovariectomized rats, examining the ability of E₂-CDS to affect LH levels. Experiment 3 examined the dose-response of LH inhibition in ovariectomized rats on day 12 post-drug treatment following a single i.v. dose of 0.1, 0.5, 2.0 or 5.0 mg/kg of E₂-CDS; of 0.07, 0.35, 1.38, 3.46 or 10.38 mg/kg estradiol; or of DMSO vehicle (0.5 mg/kg). Blood samples were collected on day 12 post-treatment. Experiment 4 compared a single i.v. dose of 0.5 mg/kg E₂-CDS with equimolar estradiol 17-valerate for LH inhibition, and blood samples were collected on day 12 or 18 post-treatment. Serum LH was decreased by 75% and 91% compared to vehicle at the 2 or 5 mg/kg dosage, respectively, of E₂-CDS, in Experiment 3. The dose of 0.5 mg/kg gave heterogeneous results and therefore was reexamined in Experiment 4. In Experiment 4, LH was significantly decreased by E₂-CDS compared to estradiol valerate. No serum estradiol analyses were done in the female tests. In the castrated male rat, a single i.v. injection of 3 mg/kg of E₂-CDS showed very high serum estradiol at early time points of 4 and 8 hours (>1000 pg/ml), and elevated levels for several days post-treatment, but not at 12, 18 and 24 days post-treatment, while LH levels remained very significantly reduced at 12, 18 and 24 days post-treatment.

Anderson et al, Life Sciences 42, 1493-1502 (1988) reported tests in ovariectomized female rats in which the rats received an intravenous injection of E₂-CDS at 10, 33, 100 or 333 μg/kg or the vehicle DMSO, 0.5 ml/kg, every two days for seven injections (2 weeks) or a single injection only 2 days before sacrifice. The single dose of E₂-CDS caused dose-dependent reduction in serum LH (39-52%) at 33 to 333 μg/kg, but no effect at 10 μg/kg. Multiple injections caused 32-76% reduction in serum LH at 10 to 333 μg/kg. Serum estradiol was reported to be unchanged at E₂-CDS doses of 10 and 33 μg/kg, increased to 21 pg/ml and 23 pg/ml in the single and multiple dose groups, respectively, at the 100 μg/kg dose, and to 59 pg/ml and 60 pg/ml for the single and multiple 333 μg/kg groups, respectively. The authors concluded that a single dose or multiple doses of E₂-CDS can reduce serum LH without elevating serum estradiol. However, serum was not collected until two days after the last injection, which in the case of multiple doses was sixteen days after the first injection. Thus, estradiol levels at early time points were not investigated and the authors' conclusion that serum LH can be reduced in ovariectomized female rats without elevating serum estradiol only related to serum estradiol values at two days after the end of the treatment period.

Rahimy et al., Maturitas 13, 51-63 (1991) reported on the effects of E₂-CDS on tail-skin temperature of the rat and its implications for menopausal hot flush. The study evaluated the effects of E₂-CDS versus E₂ on the tail-skin temperature (TST) surge associated with administration of naloxone to morphine-dependent rats. Ovariectomized rats received single or multiple i.v. doses of E₂-CDS at 1.0 mg/kg or E₂ (0.5 mg pellet) weekly for 1 to 3 weeks before temperature recording. A single 1 mg/kg i.v. injection of E₂-CDS attenuated the naloxone-induced rise in TST by 25% when tested one week after injection but that amount was not statistically significant. Multiple i.v. injections of 1 mg/kg (that is, once weekly for three weeks) significantly attenuated the naloxone-induced rise in TST when tested one week after the last injection. Multiple doses of E₂-CDS significantly elevated plasma estradiol. Plasma LH was significantly suppressed for single and multiple doses of E₂-CDS. The authors also determined plasma levels of LH and E₂ after a single i.v. 1.0 mg/kg dose of E₂-CDS to ovariectomized rats beginning 0.5 hour after drug treatment (0.5, 1, 2, 4, 8, 12, 24, 48, 96 and 168 hours post-injection). Plasma E₂ concentrations were increased to 1.9±0.08 ng/ml (1900±80 pg/ml) 30 minutes after E₂-CDS administration, then decreased by 50% at 3 hours and more than 91% at 24 hours post-treatment. The authors noted that the morphine-dependent naloxone-withdrawal rat model was to their knowledge the only animal model available to evaluate the effectiveness of E₂-CDS for treatment of hot flushes, but also questioned whether this model is actually analogous to menopausal hot flushes since in some animals (50% or less) the E₂-treatment did not completely stabilize. If the model were truly consistent with the estrogen deprivation which results in hot flushes, the performance of estrogens in the test would have been more consistent.

Moreover, no animal tests of E₂-CDS relating to female sexual dysfunction such as inhibited sexual desire have ever been reported.

Lower doses were also included as part of toxicity testing in women, as is required to establish safety. However, it was expected that dosages would need to be about 0.3 mg/kg in postmenopausal women to effectively suppress LH and treat postmenopausal symptoms long term.

Clinical studies of E₂-CDS are reported in Brewster et al., Rev. Neurosci. 2, 241-285 (1990). The first human testing was a rising dose study in menopausal women. Each of 10 subjects received a single i.v. injection of E₂-CDS dissolved in 20% (w/v) HPβCD. Blood samples were obtained at 15 minutes and 30 minutes and at 1, 2, 4, 8, 24 and 48 hours after drug administration, as well as at days 4 and 7 after administration. Doses of E₂-CDS of 10, 20, 40, 80, 160, 320 and 640 μg and 1.28 mg (0.16, 0.32, 0.71, 1.19, 2.80, 5.87, 7.57, 10.0, 19.8 and 19.7 μg/kg, respectively) were given intravenously and blood samples were analyzed for LH, FSH and 17β-estradiol (E₂). Minimal effects on plasma LH were found in the 10-40 μg dose group, threshold effects in the 80-640 μg dose group and substantial and sustained decreases in plasma LH in the 1280 μg dose group. In another study, one post-menopausal human volunteer receiving a single 1280 μg i.v. dose of E₂-CDS was found to have LH lowered in a “clinically meaningful” way through 96 hours after E₂-CDS administration. Initial serum estradiol levels for E₂-CDS, although much less than those for estradiol itself (which were too high to calculate with the standard curve), in a postmenopausal volunteer given a single 1280 μg i.v. dose, were about 1500 pg/ml, which is nevertheless very high. While treatment of menopausal symptoms such as hot flushes was the ultimate hope of clinical trials, as discussed by Brewster et al., no such treatment was in fact reported. Moreover, there was not even an allusion to treatment of female sexual dysfunction of the hypoactive sexual desire type or the sexual pain type; indeed, not even studies of E₂-CDS in an animal model for these conditions have been previously described or proposed.

Recently, the generally accepted notion that treatment of postmenopausal women with estrogen combined with progestin offered protection from coronary heart disease as well as improvement in health-related quality of life has not proved to be correct. Constant elevated peripheral exposure to estrogens may in fact lead to a number of pathological conditions, including breast cancer, coronary heart disease and pulmonary embolism; Beral et al., Lancet, 360 (9337), 942-944 (2002). Contrary to earlier expectations, hormone replacement therapy (HRT) does not lower the incidence of coronary heart disease; Low et al., Am. J. Med. Sci., 324(4), 180-184 (2002). The estrogen plus progestin combination of the Women's Health Initiative trial in postmenopausal women was stopped prematurely due to an unacceptably increased risk for invasive breast cancer, stroke and heart attack; Rossouw et al., J. Am. Med. Assoc. 288, 321-333 (2002).

It is thus apparent that there is a need for methods for treating female sexual dysfunction, including symptoms of estrogen deprivation in postmenopausal women, without elevated peripheral exposure to estrogens.

With respect to prior investigations in the male, the Anderson et al. '911 patent showed that, in castrated male rats, an amount of E₂-CDS of 3 mg/kg i.v. was typically used to provide activity for a period of 1 month. This amount stimulated mounting behavior, increased intromission behavior and reduced both mount latency and intromission latency. The conclusion was that E₂-CDS was a potent, long-acting stimulant of the proceptive components of masculine sexual behavior. The Anderson et al. patent suggested use of E₂-CDS alone if deficits in peripheral androgen-responsive tissues were not an issue; in other cases, administration together with an androgen such as testosterone was suggested. Such an amount of 3 mg/kg is generally 10 times the mg/kg amount expected to be comparable in humans. Thus, a 0.3 mg/kg amount was expected to provide comparable results in men. For an overview of prior work in the male with E₂-CDS, again see Brewster et al., Rev. Neurosci. 2, 241-285 (1990).

Despite the foregoing, it is noted that peripheral estradiol levels were not measured or reported in the Anderson et al. patent. However, Simpkins et al., J. Med. Chem. 29, 1809-1812 (1986), reported that after a single i.v. administration of 3 mg/kg E₂-CDS to castrated male rats, blood samples taken at 12-24 days after administration showed no significant differences in serum E₂ among E₂-CDS, an equimolar dose of estradiol and the DMSO vehicle. Anderson et al., Pharmacology Biochemistry & Behavior 27, 265-271 (1987) noted that only the mounting aspect of consummatory behavior was positively affected by 3 mg/kg E₂-CDS administered as a single tail vein injection in DMSO; ejaculatory behavior was not restored. That Anderson et al. article also noted that serum estradiol was not elevated in orchidectomized rats treated intravenously with 3 mg/kg E₂-CDS over controls 4 to 8 days after treatment and suggested studies combining E₂-CDS with testosterone and/or dihydrotestosterone were needed to more fully address psychological impotence coupled with deficiencies of peripheral androgens. Another report of early work with E₂-CDS appears in Estes et al., Life Sciences 40, 1327-1334 (1987), and includes more specific data for peripheral estradiol levels. In a first experiment, male rats were orchidectomized at 2-3 months of age, and two weeks later 3 mg/kg of E₂-CDS was administered to them by a single tail vein injection; blood samples were taken at 0, 4, 8, 24 and 48 hours and at 4, 8 and 12 days post-treatment. Serum estradiol levels exceeded 1000 pg/ml at 4 and 8 hours post-treatment and were still high at 24 hours (637 pg/ml) and at 2 days (285 pg/ml). In a second experiment, 12, 18 and 24 days post-drug administration, there was no significant differences found in serum estradiol among the three test groups (E₂-CDS, estradiol and DMSO control). Virtually all of these studies reported significant and prolonged (at least 24 days) suppression of LH. Anderson et al., Endocrine Research, 14 (2 & 3), 131-148 (1988), subsequently reported on the effects of E₂-CDS on serum estradiol and testosterone in middle-aged (18 months), intact male rats. In those studies, which were undertaken to investigate effects on body weight, at a dose of 1 mg/kg i.v. in DMSO, E₂-CDS increased serum estradiol levels 100-fold on day 1, and thereafter decreased; nevertheless, those levels remained 13 times initial levels at 7 days and nearly five times initial levels at 14 days. Serum testosterone levels were decreased by 99% one day after E₂-CDS treatment and remained suppressed by more than 96% through 14 days post-treatment.

Richard M. Sharpe recently published on the roles of estrogen on the male in TEM, Vol. 9, No. 9, 371-377 (1998) and emphasized that the balance in action between androgens and estrogens might be of central importance at many estrogen target sites. As examples of this importance, he cites “clover disease” in sheep, where phytoestrogens in clover caused death in castrated rams (with low circulating testosterone) but did not greatly affect intact rams; developmental abnormalities of the male reproductive system which can be caused by either an estrogen or an anti-androgen; and the fact that gynaecomastia in males can be caused by too little androgen or too much estrogen.

Even more recently, Adaikan et al., International Journal of Impotence Research 15, 38-43 (2003), note that “Sexual dysfunction with impotence and loss of libido is an important accompaniment of deranged testicular pathways and ageing in males. Diagnostically, low serum testosterone (T) level correlates with impairment of penile sensitivity, nocturnal penile tumescence and spontaneous morning erections in man and reduced cavemosal pressure response in the animal model. Besides clinical signs, the endocrine profile is further compounded by a functional excess of oestradiol (E₂) that offsets the delicate E₂-T balance.” Hypogonadism and exposure to environmental estrogens also are characterized by such hormonal imbalance. Administration of 0.1 and 0.01 mg of estradiol by oral gavage daily for 1 week (acute test) and for 12 weeks (chronic test) to groups of sexually mature male rats was found to significantly increase serum estradiol and significantly decrease serum testosterone at the higher dose (acute test) and at both doses (chronic test). The authors also found a significant prolongation of mount latency, intromission latency, and post-ejaculatory mounting latency accompanying a two- to five-fold increase in serum estradiol and simultaneous reduction of testosterone. Also at the higher dose, the ICP response to nerve stimulation was significantly impaired.

It is apparent from the foregoing that, to have a practical approach to male sexual dysfunction, particularly in the human male, it is imperative that high peripheral estradiol levels be avoided.

It is thus apparent that there is a need for methods for treating male sexual dysfunction, without elevated peripheral exposure to estrogens.

### SUMMARY OF THE INVENTION

In a first aspect of the present invention, there is provided a method for the treatment of female sexual dysfunction in a female mammal in need of such treatment, said method comprising administering to said mammal the compound 17β-[(1-methyl-1,4-dihydro-3-pyridinyl)carbonyloxy]estra-1,3,5(10)-trien-3-ol in an amount effective to diminish symptoms of said dysfunction which does not elevate average steady-state peripheral estradiol levels to above about 50-60 pg/ml.

In a second aspect of the present invention, there is provided a method for the treatment of female sexual dysfunction in a woman in need of such treatment, said method comprising administering to said woman the compound 17β-[(1-methyl-1,4-dihydro-3-pyridinyl)carbonyloxy]estra-1,3,5(10)-trien-3-ol in an amount effective to diminish or alleviate symptoms of said dysfunction which does not elevate average steady-state peripheral estradiol levels to above about 50-60 pg/ml.

In a third aspect, the invention provides a method for the treatment of postmenopausal symptoms in a postmenopausal woman in need of same, said method comprising administering to said woman the compound 17β-[(1-methyl-1,4-dihydro-3-pyridinyl)carbonyloxy]estra-1,3,5(10)-trien-3-ol in an amount effective to diminish or alleviate said symptoms which does not elevate average steady-state peripheral estradiol levels to above about 50-60 pg/ml.

In particular embodiments, the foregoing aspects of the invention further comprise repeated daily or every other day dosing of amounts as small as about 0.01 mg/kg or lower (about 0.5 to about 2.0 mg per day) in postmenopausal women) via buccal administration, and/or without elevation of average steady-state estradiol peripheral levels in such women to above about 40 pg/ml, even about 20 pg/ml or lower, and/or with average peak estradiol peripheral levels (which are reached shortly after administration) in such women not above about 70-90 pg/ml or even lower, and/or administering the active compound in cyclodextrin, particularly as a substantially saturated complex with hydroxypropyl-β-cyclodextrin (to achieve the highest degree of thermodynamic activity).

In yet another aspect of the present invention, there is provided a method for the treatment of male sexual dysfunction in a male mammal in need of such treatment, said method comprising administering to said mammal the compound 17β-[(1-methyl-1,4-dihydro-3-pyridinyl)carbonyloxy]estra-1,3,5(10)-trien-3-ol in an amount effective to diminish symptoms of said dysfunction which does not substantially elevate average peripheral estradiol levels to above average normal peripheral levels in the male mammal.

In still a further aspect of the present invention, there is provided a method for the treatment of male sexual dysfunction in a man in need of such treatment, said method comprising administering to said man the compound 17β-[(1-methyl-1,4-dihydro-3-pyridinyl)carbonyloxy]estra-1,3,5(10)-trien-3-ol in an amount effective to diminish or alleviate symptoms of said dysfunction which does not substantially elevate average peripheral estradiol levels to above average normal levels in men.

In particular embodiments, the foregoing aspects of treating male sexual dysfunction further comprise repeated daily or every other day dosing of amounts comparable to 0.01 mg/kg i.v. in the castrated male rat, for example, amounts of about 0.01 to about 0.5 mg per day buccally to men, for such period of time as required until symptoms diminish (for example, approximately 2 to 7 days in men), with resumption of daily or every other day dosing when symptoms recur, and/or administering the active compound in cyclodextrin, particularly as a substantially saturated complex with hydroxypropyl-β-cyclodextrin (to achieve the highest degree of thermodynamic activity).

### BRIEF DESCRIPTION OF THE DRAWINGS

Other details of the invention will be apparent from the following detailed description and accompanying drawings, in which the Figures are as follows.

FIG. 1 is a plot of lordosis quotient (percent responders) versus time in days for varying doses of estradiol-CDS, i.e., 17β-[(1-methyl-1,4-dihydro-3-pyridinyl)carbonyloxy]estra-1,3,5(10)-trien-3-ol (E₂-CDS), at 0.003 mg/kg (Δ), 0.01 mg/kg (♦), 0.03 mg/kg (●), and of the control vehicle, hydroxypropyl-β-cyclodextrin (HPβCD) solution (▪), in ovariectomized female rats after daily intravenous (i.v.) injections for five days, with observations beginning on day 3 following the first injection.

FIG. 2 is a plot of lordosis quotient (percent responders) versus time in days for varying doses of estradiol benzoate, at 0.003 mg/kg (Δ), 0.01 mg/kg (⋄) and 0.03 mg/kg (◯), and of the control vehicle, hydroxypropyl-β-cyclodextrin (HPβCD) solution (□), in ovariectomized female rats after daily intravenous (i.v.) injections for five days, with observations beginning on day 3 following the first injection.

FIG. 3 is a group of three (3) plots of lordosis quotient (percent responders) versus time in days for the same doses as in FIGS. 1 and 2, but grouped so as to compare the same doses of E₂-CDS and estradiol benzoate.

FIG. 4 is a plot of LH levels in ng/ml plasma versus time in days for varying doses of E₂-CDS at 0.003 mg/kg (●), 0.01 mg/kg (▴), 0.03 mg/kg (▪), and of the control (♦) in ovariectomized female rats after daily single i.v. tail injections for five days, with observations beginning on day 3 following the first injection.

FIG. 5 is a plot of LH levels in ng/ml plasma versus time in days for varying doses of estradiol benzoate at 0.003 mg/kg (●), 0.01 mg/kg (▴), 0.03 mg/kg (▪), and of the control (♦) in ovariectomized female rats after daily single i.v. tail injections for five days, with observations beginning on day 3 following the first injection.

FIG. 6 is a bar graph illustrating the effect of varying doses of estradiol-CDS (E₂-CDS), at 0.03 mg/kg ( [symbol] ), 0.3 mg/kg ( [symbol] ), 3.0 mg/kg (▪), and of the control vehicle, hydroxypropyl-β-cyclodextrin (HPβCD) solution (□), on the mounting performance (% responders) in intact male rats, and in castrated male rats at days 0, 3, 7, 14, 21, 28 and 35, after a single intravenous (i.v.) injection.

FIG. 7 is a bar graph illustrating the effect of varying doses E₂-CDS, at 0.03 mg/kg ( [symbol] ), 0.3 mg/kg ( [symbol] ) and 3.0 mg/kg (▪) and of the control vehicle, HPβCD (□), on the intromission percentage (% responders) in intact male rats, and in castrated male rats at days 0, 3, 7, 14, 21, 28 and 35 after a single intravenous (i.v.) injection.

FIG. 8 is a bar graph and accompanying chart illustrating the effect of varying doses of E₂-CDS, at 0.03 mg/kg ( [symbol] ), 0.3 mg/kg ( [symbol] ), and 3.0 mg/kg (▪) and of the control vehicle HPβCD (□), on the mounting frequency in intact male rats, and in castrated male rats at days 0, 3, 7, 14, 21, 28 and 35 after a single intravenous (i.v.) injection.

FIG. 9 is a bar graph and accompanying chart illustrating the effect of varying doses of E₂-CDS, at 0.03 mg/kg ( [symbol] ), 0.3 mg/kg ( [symbol] ) and 3 mg/kg (▪), and of the control vehicle HPβCD (□), on the mounting latency, in minutes, in intact male rats, and in castrated male rats at days 0, 3, 7, 14, 21, 28 and 35 after a single intravenous (i.v.) injection.

FIG. 10 is a bar graph and accompanying chart illustrating the effect of varying doses of E₂-CDS, at 0.03 mg/kg ( [symbol] ), 0.3 mg/kg ( [symbol] ) and 3 mg/kg (▪), and of the control vehicle HPβCD (□), on the intromission frequency in intact male rats, and in castrated male rats at days 0, 3, 7, 14, 21, 28 and 35 after a single intravenous (i.v.) injection.

FIG. 11 is a bar graph and accompanying chart illustrating the effect of varying doses of E₂-CDS, at 0.03 mg/kg ( [symbol] ), 0.3 mg/kg ( [symbol] ) and 3 mg/kg (▪) and of the control vehicle HPβCD (□), on the intromission latency, in minutes, in intact male rats, and in castrated male rats at days 0, 3, 7, 14, 21, 28 and 35 after a single intravenous (i.v.) injection.

FIG. 12 is a plot of LH levels in ng/ml plasma versus time in days for varying doses of E₂-CDS at 0.03 mg/kg (x), 0.3 mg/kg (♦) and 3 mg/kg (Δ) and of the control vehicle HPβCD (●) in orchidectomized (castrated) male rats for a period of 35 days after a single intravenous (i.v.) injection).

FIG. 13 is a bar graph illustrating the effect of 0.03 mg/kg ( [symbol] ) E₂-CDS administered i.v. once, and 0.01 mg/kg ( [symbol] ) E₂-CDS administered i.v. once daily for 10 days, and the control vehicle, HPβCD (□), on the mounting performance (% responders) in intact male rats, and in castrated male rats at days 0, 1, 3, 7, 14 and 21.

FIG. 14 is a bar graph illustrating the effect of 0.03 mg/kg ( [symbol] ) E₂-CDS administered i.v. once, and 0.01 mg/kg ( [symbol] ) E₂-CDS administered i.v. once daily for 10 days, and the control vehicle, HPβCD (□), on the intromission performance (% responders) in intact male rats, and in castrated male rats at days 0, 1, 3, 7, 14 and 21.

FIG. 15 is a bar graph and accompanying chart illustrating the effect of 0.03 mg/kg ( [symbol] ) E₂-CDS administered i.v. once, and 0.01 mg/kg ( [symbol] ) E₂-CDS administered i.v. once daily for 10 days, and the control vehicle, HPβCD (□), on the mounting frequency (number of mounts), in intact male rats, and in castrated male rats at days 0, 1, 3, 7, 14 and 21.

FIG. 16 is a bar graph and accompanying chart illustrating the effect of 0.03 mg/kg ( [symbol] ) E₂-CDS administered i.v. once, and 0.01 mg/kg ( [symbol] ) E₂-CDS administered i.v. once daily for 10 days, and the control vehicle, HPβCD(□), on the mounting latency, in minutes, in intact male rats, and in castrated male rats at days 0, 1, 3, 7, 14 and 21.

FIG. 17 is a bar graph and accompanying chart illustrating the effect of 0.03 mg/kg ( [symbol] ) E₂-CDS administered i.v. once, and 0.01 mg/kg ( [symbol] ) E₂-CDS administered i.v. once daily for 10 days, and the control vehicle, HPβCD(□), on the intromission latency, in minutes, in intact male rats, and in castrated male rats at days 0, 1, 3, 7, 14 and 21.

FIG. 18 is a bar graph and accompanying chart illustrating the effect of 0.03 mg/kg ( [symbol] ) E₂-CDS administered i.v. once, and 0.01 mg/kg ( [symbol] ) E₂-CDS administered i.v. once daily for 10 days, and the control vehicle, HPβCD (□), on the intromission frequency (number of intromissions) in intact male rats, and in castrated male rats at days 0, 1, 3, 7, 14 and 21.

FIG. 19 is a plot of LH levels in ng/ml plasma versus time in days for a dose of 0.03 mg/kg (x) E₂-CDS administered i.v. once, a dose of 0.01 mg/kg (●) E₂-CDS administered i.v. once daily for 10 days and of the control vehicle HPβCD(◯) in orchidectomized (castrated) male rats for a period of 14 days.

### DETAILED DESCRIPTION OF THE INVENTION

Throughout the instant specification and claims, the following definitions and general statements are applicable.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

The term “complex” as used herein means an inclusion complex, in which a hydrophobic portion of the E₂-CDS molecule (typically a portion of the steroidal ring system) is inserted into the hydrophobic cavity of the cyclodextrin molecule. For example, in the case of E₂-CDS and HPβCD, it is believed that in the 1:1 complex, the aromatic A ring of the steroid is included. At higher HPβCD concentrations, a 1:2 complex of E₂-CDS:HPβCD forms and the second HPβCD molecule may interact with the dihydronicotinate group in the E₂-CDS molecule.

As used herein, whether in a transitional phrase or in the body of a claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a composition, the term “comprising” means that the composition includes at least the recited features or components, but may also include additional features or components.

The terms “consists essentially of” or “consisting essentially of” have a partially closed meaning, that is, they do not permit inclusion of steps or features or components which would substantially change the essential characteristics of a process or composition; for example, steps or features or components which would significantly interfere with the methods described herein, i.e., the methods are limited to the specified features and those which do not materially affect the basic and novel characteristics of the invention. The basic and novel features herein are the provision of methods for treating male and female sexual dysfunction in which the amount of E₂-CDS administered is carefully controlled so that it diminishes symptoms in an amount which does not elevate average steady-state peripheral estradiol levels to above average normal peripheral levels in the male, or which does not elevate average steady-state peripheral estradiol levels to above about 50-60 pg/ml in the female. In particular embodiments, the methods comprise buccal administration of an anhydrous formulation of a substantially saturated complex of the compound 17β-[(1-methyl-1,4-dihydro-3-pyridinyl)carbonyloxy]estra-1,3,5(10)-trien-3-ol with a hydroxyalkyl, carboxyalkyl or carboxymethylethyl derivative of β- or γ-cyclodextrin comprising from about 0.01 to about 0.5 mg of said compound per day in men and from about 0.5 to about 2.0 mg of said compound per day in women.

The terms “consists of” and “consists” are closed terminology and allow only for the inclusion of the recited steps or features or components.

As used herein, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

The term “saturated” when used in conjunction with a complex of E₂-CDS in cyclodextrin means that the complex is saturated with the E₂-CDS, that is, the complex contains the maximum amount of the E₂-CDS which can be complexed with a given amount of cyclodextrin under the conditions of complexation used. A phase solubility study can be used to provide this information. Alternatively, a saturated complex may be arrived at empirically by simply adding the E₂-CDS to an aqueous solution of the selected cyclodextrin until a precipitate (of uncomplexed E₂-CDS) forms; ultimately, the precipitate is removed and the solution lyophilized to provide the dry saturated complex.

The term “substantially”, as in “substantially saturated” means that from 80% to 100%, preferably from 90% to 100%, of the complex is in saturated form. In any other context, “substantially” similarly means within 20% of the exact calculated amount, preferably within 10% of that amount. Whatever the context, within 5% of the calculated amount is most desirable.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001).

The expression “peripheral estradiol levels” as used herein refers to serum estradiol levels obtained throughout the treatment period, using repeated dosing on a once per day or every other day schedule.

The expression “steady-state peripheral estradiol levels” as used herein refers to serum estradiol levels obtained throughout the treatment period, using repeated dosing on a once per day or every other day schedule, excluding initial peak levels obtained within 1-2 hours after the initial dose.

The meanings of the expressions “male sexual dysfunction”, “female sexual dysfunction” and “postmenopausal symptoms” are well-known and are explained in the foregoing Background of the Invention.

The expression “lordosis” as used herein refers to vertebral dorsiflexion performed by female quadrupeds in response to adequate stimuli from a reproductively competent male. The lordosis quotient is calculated as 100×number of lordoses/10 mounts.

The expressions “mounting” and “intromission” are explained in the foregoing Background of the Invention.

The expression “male mammal” is intended to include any male mammal but especially human beings, domestic and farm animals, zoo animals and rare or endangered or expensive mammalian species.

The expression “female mammal” is intended to include any female mammal but especially human beings, domestic and farm animals, zoo animals and rare or endangered or expensive mammalian species.

The active ingredient in the instant methods and formulations, E₂-CDS, i.e. 17β-[(1-methyl-1,4-dihydro-3-pyridinyl)carbonyloxy]estra-1,3,5(10)-trien-3-ol, is well-known, and methods for its synthesis and its complexation with cyclodextrin has been described in numerous patents and non-patent publications, including the various Bodor, Bodor et al. and Anderson et al. patents referred to in the foregoing Background of the Invention.

### Preparation of Apporximately 3% Complex of E₂-CDS with HPβCD

Dissolve 232 g of 2-hydroxypropyl-β-cyclodextrin (HPβCD) (Cerestar, degree of substitution 4.5) in deionized 465 mL water (ASTM Type I) to form an approximately 33% w/v solution. Adjust the pH to 8.4-9.6 with sodium carbonate 1% solution. Degas the solution by passing argon through it. Add slowly, drop-wise, under stirring and bubbling argon, at 20-25° C., a solution of E₂-CDS (7.5 g) in ethanol (188 mL). Allow time after each addition for the solution to become clear. The addition takes about 4 hours and it is slower at the end. A clear solution will result. Evaporate the solution to dryness in a rotary evaporator (bath temperature 35° C.). Reconstitute the residue in water, calculated to obtain the initial concentration of the cyclodextrin solution. Filter the solution through a 47 mm, 0.45 μm nylon 66 membrane filter, while covering with argon. Freeze-dry the filtrate, grind the resulting solid in a blender and pass it through a 60 mesh sieve. The resulting complex, an off-white amorphous solid (˜233 g), is transferred in a jar and analyzed. The complex should contain about 29-32 mg E₂-CDS per gram. E₂-CDS should have a chromatographic purity of at least 97% by HPLC. The yield of complexation (based on E₂-CDS) should be 82-96%.

### Preparation of Approximately 2.5% Complex of E₂-CDS with HPγCD

Dissolve 45 g of 2-hydroxypropyl-γ-cyclodextrin (HPγCD) (Wacker, Cavasol W8 HP) in deionized 135 mL water (DIUF) to form an approximately 25% w/v solution. Adjust the pH to 8.4-9.6 with sodium carbonate 1% solution. Degas the solution by passing argon through it. Add slowly, drop-wise, under stirring and bubbling argon, at 20-25° C., a solution of E₂-CDS (1.5 g) in ethanol (3 mL). Allow time after each addition for the solution to become clear. The addition takes about 4 hours and it is slower at the end. A clear solution will result. Evaporate the solution to dryness in a rotary evaporator (bath temperature 35° C.). Reconstitute the residue in water, calculated to obtain the initial concentration of the cyclodextrin solution. Filter the solution through 47 mm, 0.45 μm nylon 66 membrane filter, while covering with argon. Freeze-dry the filtrate, grind the resulting solid in a blender and pass it through a 60 mesh sieve. The resulting complex, an off-white amorphous solid (˜42 g), is transferred in a jar and analyzed. The complex should contain about 20-25 mg E₂-CDS per gram. E₂-CDS should have a chromatographic purity of at least 97% by HPLC. The yield of complexation (based on E₂-CDS) should be 82-96%.

### Preparation of Complex E₂-CDS with CMEβCD

Method A:

Dissolve 100 mg of E₂-CDS and 500 mg of O-carboxymethyl-O-ethyl-β-cyclodextrin (CMEβCD) in 10 mL of ethanol and sonicate the solution for 1 hour. Then remove the solvent, reconstitute the residue with water, filter and lyophilize. The complex should contain about 25 mg E₂-CDS/g.

Method B:

Dissolve 2 g of CMEβCD in 20 mL of 0.10M pH 9.0 borate buffer. Adjust the pH with 1N sodium hydroxide solution. Then dissolve 150 mg of E₂-CDS in 2 mL of ethanol and add the resultant solution to the cyclodextrin solution. Stir for 3 hours at 0° C. under argon, remove the solvent in vacuo, reconstitute the residue with pH 9 borate buffer and lyophilize.

### Manufacture of Buccal Tablets for Clinical Trials

In accord with the invention, a buccal tablet was designed for use in clinical trials to deliver E₂-CDS transmucosally and thus avoid the instability of E₂-CDS in gastrointestinal fluid, which leads to multiple decomposition productions starting with water addition and/or oxidation, as well as hepatic first pass metabolism. Transmucosal absorption is highly effective from the invention's saturated complex of E₂-CDS in HPβCD (as prepared, for example, in EXAMPLE 2 above) with minimal additives. A placebo was also prepared for the clinical trials.

FORMULATION

| | | | E₂-CDS | | E₂-CDS | | E₂-CDS | |
|---|---|---|---|---|---|---|---|---|
| | Placebo | | 0.5 mg | | 1.0 mg | | 2.0 mg | |
| E₂-CDS/ | 0.00 | | 16.67 | | 33.33 | | 66.67 | |
| HPβCD | | | | | | | | |
| complex | | | | | | | | |
| HPβCD | 33.33 | | 0.00 | | 0.00 | | 0.00 | |
| (freeze-dried) | | | | | | | | |
| Sorbitol | 62.67 | | 82.33 | | 65.67 | | 32.33 | |
| powder NF | | | | | | | | |
| Magnesium | 1.00 | | 1.00 | | 1.00 | | 1.00 | |
| stearate NF | | | | | | | | |
| Opadry Yellow | 3.00 | | 0.00 | | 0.00 | | 0.00 | |
| 31F22300 | | | | | | | | |
| | 100.00 | mg | 100.00 | mg | 100.00 | mg | 100.00 | mg |

Similar buccal tablets can be prepared containing complexes of E₂-CDS with other cyclodextrins such as HPγCD, CMEβCD or other cyclodextrin identified in this specification.

### Investigation of Female Rat Sexual Behavior After Overiectomy

Rationale

Castration causes the termination of sexual behavior in rats, but the sexual activity of castrated female rats can be reestablished by administration of estradiol.

In female rats, estradiol acts in the hypothalamus and preoptic area to regulate the expression of lordosis, an important component of female reproductive behavior and a characteristic posture of the female for a sexually active male to allow copulation. Estradiol acts on multiple molecular targets that may converge on common biochemical pathways to ensure integration of sensory and neurochemical cues that regulate lordosis expression. Thus, lordosis was selected as an indicator of restoration of female sexual function in ovariectomized female rats and an appropriate indicator for alleviating symptoms of female sexual dysfunction.

Circulating luteinizing hormone (LH) is a biomarker reflecting the CNS effects of estradiol. Estrogen diminishes the secretion of luteinizing hormone-releasing hormone (LHRH) and hence reduces the secretion of LH. Therefore, LH and estradiol levels were investigated to measure the central and peripheral effects of E₂-CDS, respectively.

Experimental Design

Adult female Sprague Dawley rats (220-250 g) from Charles River Hungary Ltd., Godollo, Hungary, were used. Animals were kept in community cages (4 animals/cage) in a climate-controlled room (23±2° C., 50-60% humidity), with a 14 hour light, 10 hour dark cycle of artificial lighting, using reversed light/dark cycle. Food and water were available ad libitum.

After a minimum five-day acclimatization period, animals were ovariectomized under ether anesthesia, then were left to recover for 3 weeks before testing (reconvalescence). All animals were treated in accordance with the guidelines of the European Communities Council Directive (86/609/EEC) and studies were permitted by the Institutional Animal Care Commission.

Estradiol benzoate and progesterone were obtained from Sigma Chemical Co. Inc., Budapest, Hungary. 2-Hydroxypropyl-β-cyclodextrin was purchased from Cerestar Inc., Hammond, Ind., U.S. Estradiol benzoate was dissolved in 40 w/v% 2-hydroxypropyl-β-cyclodextrin (HPβCD) solution and diluted with 27 w/v % HPβCD solution (0.29 mg/kg is equimolar to that of 0.3 mg/kg E₂-CDS). E₂-CDS as a 3% complex with HPβCD (E₂-CDS-CD) was dissolved in distilled water and diluted with 27% HPβCD solution. E₂-CDS-CD was synthesized by Alchem Laboratories Corporation, Alachua, Fla., US.

Behavioral Testing

After recovery from surgery, ovariectomized female rats were divided into four groups and treated once a day for five days intravenously, via a bolus injection through the tail vein, as follows: (1) control, 27% HPβCD solution; (2) 0.003 mg/kg E₂-CDS dissolved in 27% HPβCD solution; (3) 0.01 mg/kg E₂-CDS dissolved in 27% HPβCD solution; and (4) 0.03 mg/kg E₂-CDS dissolved in 27% HPβCD solution. A minimal number of ovariectomized (8 to 12) females were used per group. Intravenous treatments either with E₂-CDS or HPβCD (controls) were carried out daily for 5 days beginning 2 days prior to the first day of behavior observations, in a volume of 0.05 ml/100 g body weight.

The investigation of estradiol benzoate (EB) was performed in newly randomized previously ovariectomized females after a resting period of 3 weeks. Animals (7 to 11 per group) were treated with 0.003, 0.01 and 0.03 mg/kg estradiol benzoate intravenously once a day for 5 consecutive days similarly to the protocol applied for E₂-CDS. Estradiol benzoate was dissolved in 40% HPβCD and diluted with 27% HPβCD solution (0.29 mg/kg stock solution equimolar to that of E₂-CDS).

The behavior test was conducted in a plexiglass observation cage during the dark cycle. During behavioral observations, only a dim red light was on.

An experienced and active male rat was placed in the arena 5 minutes prior to the female. Each female was observed for the time of ten successful mounts per test session or for a maximum of 10 minutes, and the number of lordosis responses was recorded. The lordosis quotient (LQ) expresses the estrogen effect on sexual receptivity and was calculated as follows:
LQ=100×number of lordoses/10 mounts
The observation of the sexual behavior of each female was carried out, in the case of E₂-CDS, every day for 22 days; in the case of EB, investigations were carried out every day for 10 days. On days 0, 3, 7, 10, 12, 15 and 18, blood samples were taken to determine levels of LH and estradiol. Citrated blood samples were taken by retro-orbital sinus puncture under light ether anesthesia. The samples were stored at 4° C. for one hour, then centrifuged at 1000 g for 10 minutes. Plasma was separated and stored at −80° C. until assayed. Plasma LH concentrations from individual samples were measured by double antibody radioimmunoassay kits obtained from Amersham Pharmacia Biotech, Rome, Italy. Plasma estradiol levels were determined by double antibody I¹²⁵ isotope-RIA kits obtained from BioChem Immuno System. The limit of detection was 15 pg/ml.

Behavioral changes were analyzed using the Mann-Whitney U test (Siegel, Nonparametric Statistics for the Behavioral Sciences, New York; McGraw-Hill Book Company, Inc., 1956). The Fisher exact test was used for percentage comparisons (Zar, Biostatistical Analysis, Prentice Hall, Inc., Englewood Cliffs, N.J., 1974). Serum LH data were analyzed for each time and treatment group by analysis of variance (ANOVA) followed by Bonferroni posthoc test. Plasma LH and estradiol concentrations were evaluated by the computerized standard curve program of Prism software (Version 3.0, Graph Pad, San Diego, Calif., US).

Results

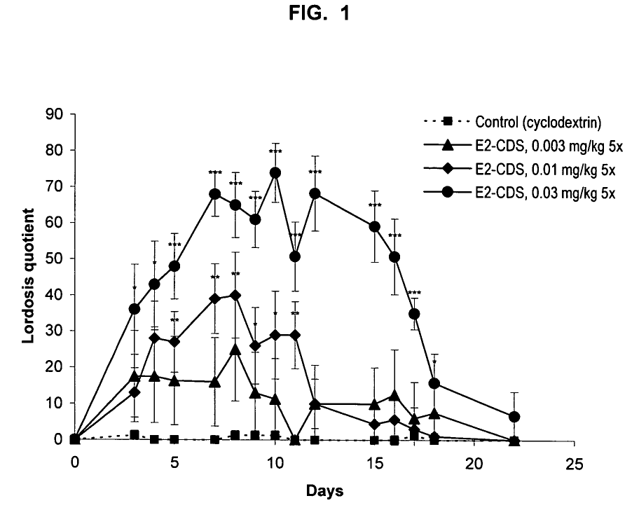

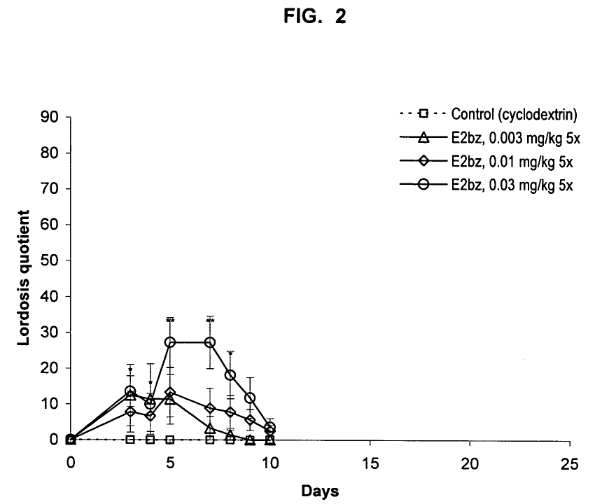

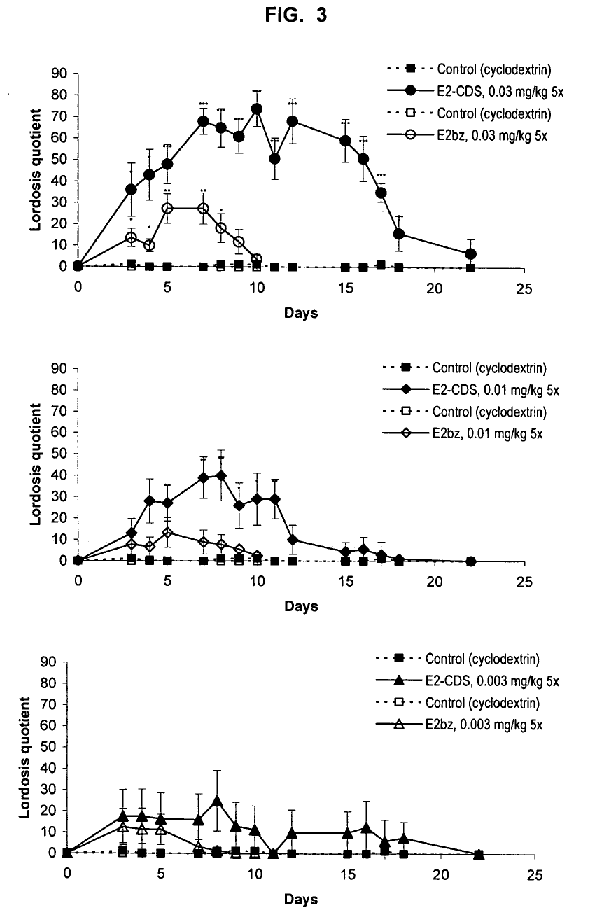

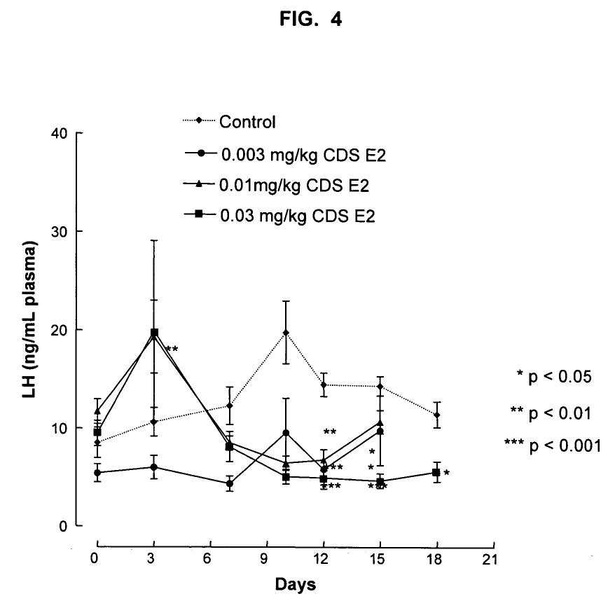

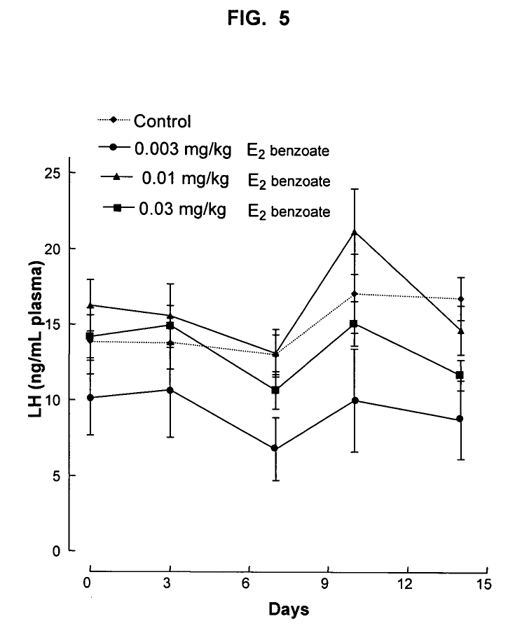

FIGS. 1-5 show the results obtained. In FIG. 1, data are mean±SE for 8-12 animals per group; *p<0.05, **p<0.01, ***p<0.001 using the Mann-Whitney U test. In FIG. 2, data are mean±SE for 7-11 animals per group, with *, ** and *** as defined for FIG. 1. The data presented in FIG. 1 and FIG. 2 are reorganized in FIG. 3 so as to more readily compare the effect of the same dose of E₂-CDS and estradiol benzoate (E₂-Benz). In FIGS. 4 and 5, data are mean±SE for 7-12 animals per group, *p<0.05, **p<0.01, ***p<0.001 using ANOVA followed by the Bonferroni posthoc test.

At the dose of 0.03 mg/kg, the lordosis quotient LQ was significantly enhanced by both E₂-CDS and estradiol benzoate. In the case of E₂-CDS, this effect lasted from day 3 to day 18, as shown in FIG. 1. The effect from estradiol benzoate was less pronounced and lasted only from day 3 to day 8; see FIG. 2. As also seen in FIG. 2 as well as the first portion of FIG. 3, the LQ value for estradiol benzoate was about three times lower than that obtained for E₂-CDS: the maximal values of LQ after E₂-CDS and estradiol benzoate treatments were 73 and 27.3 respectively.

At the dose of 0.01 mg/kg, E₂-CDS significantly enhanced the LQ from day 5 to day 11. The increase, although thereafter not statistically significant, lasted till day 15. This dose of estradiol benzoate slightly increased the LQ from day 3 to day 10 (about 3 times less compared to E₂-CDS), but this effect was not statistically significant. See FIGS. 1, 2 and the second portion of FIG. 3.

At the dose of 0.003 mg/kg, doses of the test compounds slightly enhanced the lordosis quotient, but these effects were not statistically significant (estradiol benzoate, days 3-7; E₂-CDS, days 3-18). See FIGS. 1, 2 and the third portion of FIG. 3.

FIG. 4 shows that plasma LH levels were suppressed at all dosage levels of E₂-CDS tested, i.e. at 0.003, 0.01 and 0.03 mg/kg. Even at the low i.v. dose of 0.03 mg/kg, the plasma LH level was suppressed in a statistically significant manner for up to 18 days; plasma LH suppression lasted for up to 15 days even for the very low dose of 0.003 mg/kg. In contrast, as shown in FIG. 5, none of the tested dosages of estradiol benzoate gave statistically significant LH suppression.

The foregoing studies show that E₂-CDS can restore female sexual function in rats and indicate that symptoms of female sexual dysfunction can be alleviated through its administration to females, including women, at doses far lower than previously thought possible, while maintaining appropriate peripheral levels of estrogen.

### Clinical Studies

Recently, E₂-CDS has been studied in clinical trials of postmenopausal women given a single 2.5 mg or 5 mg dose of E₂-CDS administered buccally. Even more recently, in a Phase I clinical study of postmenopausal women, two different administration regimens of a 2.86 mg E₂-CDS buccal delivery tablet were evaluated for safety and effects on hormone levels. The subjects were 12 healthy postmenopausal volunteers, divided into two groups of six. In Group A, women were dosed once daily for 10 days (10 doses); in Group B, women were dosed once every other day for 13 days (7 doses). In both groups, measurements of serum total and free estradiol, estrone, LH, FSH, prolactin, SHBG and testosterone were made at certain intervals throughout the treatment period and also at 72 hours after the last dose and levels of urinary estrone and the ratio of 2OHE₁/16OHE₁ on Day 1 and 72 hours after the last dose were determined, too. A brief evaluation of the results follows:

Results

1. Dissolution

E₂-CDS was administered in a buccal delivery form (a buccal tablet) as a saturated complex with hydroxypropyl-β-cyclodextrin. The median buccal dissolution time (and “buccal residence time”) was 11 minutes and 13 seconds (minimum 1.12 min.sec, maximum 23.03 min.sec). This dissolution time is convenient for patients.

2. Estradiol (E₂)

During the first 24 hours after the administration of 2.86 mg E₂-CDS, the maximum concentration (Cₘₐₓ) of E₂ in serum was 102±20.2 pg/mL (with subject 12, who subsequently showed much higher levels than all other subjects), and this peak was reached at 1.2±0.4 hours. The Cₘₐₓ without subject 12 was 97.8±20.0 pg/mL. The average Cₘₐₓ in the earlier clinical trial, which used a buccal delivery form with an average 45 minute dissolution rate, was 153.4 pg/mL after 2.5 mg E₂-CDS, with Tₘₐₓ of 2 hours. One explanation for this difference might be the difference in the dissolution (and buccal residence) time of the two formulations used in these two different studies.

Neither of the administration regimens (once daily, versus once every other day) resulted in an accumulation, i.e. increase, in the trough serum estradiol levels (CTR), measured always before the next consecutive dose of E₂-CDS. However, the serum levels that were established during the repeated dosing were different between the two administration regimens. At steady state E₂ CTRₘₐₓ of 95.3±76.6 pg/mL was reached with the daily administration (if values for subject 12 are omitted, this concentration is 65.2±23.2 pg/mL). The steady state CTRₘₐₓ serum concentration of E₂ was 26.4±9.8 pg/mL with the every other day administration regimen.

The post-study (72 hours after the last dose) E₂ concentration was 11.5±2.7 pg/mL in the every other day group, and 36.8±54.6 pg/mL in the once daily group, respectively. In the once daily group, this post-study value would be 12.5±6.5 pg/mL if the values for subject 12 are omitted.

3. Estrone (E₁)

E₁ was measured during the first 24 hours along with E₂ and at post-study (i.e. 72 hours after the last dose). The post-study values were 47.5±49.7 pg/mL (without subject 12: 27.8±12.8 pg/mL) and 31.4±9.4 pg/mL in the once daily, and in the every other day dosing regimen group, respectively. During repeated administration a similar trough level pattern to E₂ without accumulation can be anticipated for E₁ as well in both dosing regimen groups, i.e. a steady state at somewhat higher level for the once daily administration group, than for the once every other day group.

4. LH Suppression

During the first 24 hours the maximum decrease in LH was 13.8±4.9 and 12.7±6.8 mIU/mL from baseline in Group A and B, respectively (Group A showed slightly higher baseline values). This corresponds to a 35-40 % decrease in LH levels from baseline. The maximum LH depression occurred at 7.3±5.3, and 7.3±2.7 hours post-dose in Group A and B, respectively. At post-study (72 hours after the last dose) LH levels were not different any more from screening/baseline values. Though the 24-hour LH suppression profile was determined only during the first 24 hour post-dose period, a similar daily LH suppression pattern can be anticipated on each dosing day. Blood samples are available for additional pre-dose LH measurements for days 3-11 in group A (once daily), and for days 3, 5, 7, 9, 11, 13, in group B (every other day), respectively.

5. FSH Suppression

A 15 and 16% suppression in FSH levels was observed during the first 24 hours post-dose in Group A and B, respectively. The maximum suppression occurred at 13.2±5.7, and 11.8±5.8 hours post-dose in Group A and B, respectively. In contrast to LH, post-study FSH levels were still below the screening/baseline values (by 14-25%). The kinetics of FSH suppression seems to be different from that of LH suppression: it develops more slowly after the administration of E₂-CDS, and FSH remains somewhat suppressed throughout the entire length of the study, even at 72 hours after the last dose. Blood samples for additional pre-dose hormone level measurements are available for days 3-11, and 3, 5, 7, 9, 11, 13, in group A and B, respectively. The extent of maximum FSH suppression (12.5%) during the first 24 hour post-dose in the previous clinical study after 2.5 mg E₂-CDS was similar to the extent in this study, having in mind the slightly higher dose (2.86 mg) administered in the second study.

6. Prolactin

Mean baseline concentrations of prolactin were higher in Group A than in Group B. Likewise, there were higher mean concentrations on day 13 in Group A than on day 16 in Group B. The increase in prolactin levels compared to baseline was 29.8 and 16%, in Group A and B, respectively, by the end of the study. The differences between the two groups were not statistically significant.

7. SHBG (Sex Hormone-Binding Globulin)

SHBG concentrations in Group A (day 13) and Group B (day 16), respectively, were by 22.2 and 41.2% higher than at baseline on day 1. Statistical differences between the groups were not demonstrated.

8. Testosterone

Serum concentrations of testosterone on day 1 decreased both in Group A and B. Mean AUC₂₄ were by 31.1 and 23.0% lower than baseline AUC₂₄ (=C₀*24), in Group A and B, respectively. Serum testosterone concentrations on day 1 decreased to 1.3±1.9 and 4.0±3.9 ng/dL from 22.5±21.0 and 24.0±14.0 ng/dL, in Group A and B, respectively. The time to reach these minimum testosterone levels on day 1 were 6.5±11.7 and 7.3±11.3 hours in Group A and B, respectively. 72 hours after the last administered dose, testosterone levels returned and slightly exceeded those of baseline values by 14 and 28% in Group A and B, respectively. However, the differences between the two groups did not reach statistical significance in any parameter.

9. Urinary Estrone (E₁) and 2OHE₁/16OHE₁ (2-hydroxyestrone/16-hydroxyestrone)

Urine was collected for 24 hours on day 1 in both groups, and overnight (8 hours) on day 10 (Group A) and on day 13 (Group B), respectively, to determine the amounts of voided urinary estrone (E₁), 2OHE₁, 16OHE₁ and the ratios of 2OHE₁ to 16OHE₁. The mean amounts of E₁ and the ratios E₁/creatinine in the 24-hour urine on day 1 in both groups were very similar. Mean amounts and ratios of 2OHE₁ to 16OHE₁ in 8-hour urine on day 10 in Group A appeared slightly higher than in Group B on day 13. Consequently, the differences of mean amounts (adjusted to an 8-hour urine collection period) and differences of mean ratios of day 10-1 in Group A were higher than the corresponding differences of day 13-1 in Group B (0.15 vs. 0.05). The difference of ratios approached statistical significance (p=0.077). On the last dosing day (day 10 in Group A, day 13 in Group B, respectively) mean amounts of 2OHE₁ were 4.46-times and 2.34-times higher than those values (adjusted to an 8-hour urine collection period) on day 1 in Group A and B, respectively. However, the increases in the amounts of urinary 16OHE₁ were only 2.48, and 1.26-times higher in Group A and B, respectively at the end of the treatment period compared to the day 18-hour adjusted values. During treatment the ratios of 2OHE₁ to 16OHE₁ increased by 63.6 and 54.7% in Group A and B, respectively.

10. Safety and Tolerance

There were seven adverse events (AEs) in total experienced by four subjects. The AEs were increased SGOT and CPK levels (1-1 case) headaches (2 cases) and 1-1 cases of glossitis, nausea and vomiting. All AEs were mild or moderate, no serious AE was observed. The relationship to trial drug was judged to be reasonably attributable in the single case of glossitis. All other AEs were considered as not reasonably attributable to the trial drug. The abnormal laboratory findings were a consequence of accidental injury and values returned to normal after 7 days. One AE (headache) required treatment with a single dose of 500 mg paracetamol. All AEs resolved without sequelae.

Conslusions:

The aim of this clinical study was to collect PK data on serum hormone levels (focus on serum E₂ concentrations) during a repeated administration study. 2.86 mg E₂-CDS was administered buccally once daily (group A), or once in every other day (group B). After reaching a steady state concentration (65.2±23.2 pg/mL without subject 12 in Group A and 26.4±9.8 pg/mL in Group B, respectively), trough E₂ levels did not increase with time, there were no signs of accumulation in either of the two groups. Based on a repeated measure ANOVA of E₂ trough concentrations that did not show a significant effect of time, or a subject*time interaction between days 7-11 in Group A, and including days 5, 7, 9, 11, and 13 in Group B, it can be concluded that the steady state E₂ trough concentrations were attained by day 5 and 7 in Group B and A, respectively. The attained steady state peripheral E₂ concentration in group A was stabilized in a range (65.2±23.2 pg/mL) where clinical efficacy, i.e. relief of vasomotor and urogenital symptoms would be expected. However, bearing in mind that the mechanisms of vasomotor symptoms are mostly CNS mediated, and also based on the preclinical observations that E₂ is trickled down from the brain as it is released from the inactive E₂Q⁺ precursor trapped behind the BBB, clinical efficacy is expected also in group B at lower peripheral trough E₂ levels. For practical reasons an every other day dosing regimen might be complicated for patients, however the once daily administration with lower doses (0.5, 0.75, 1.0, 1.25, 1.5, 1.75, 2.0 mg) should be sufficient to ameliorate postmenopausal symptoms, especially vasomotor and urogenital symptoms, and to effectively treat female sexual dysfunction, especially that involving deficiencies in sexual desire or sexual pain disorders. Further, a four week pack of tablets analogous to those typically used for dispensing estrogen/progestin combinations, e.g. Prempro®, or oral contraceptives, could be used in either case, with the alternate day regimen simplified for patients by alternating E₂-CDS buccal tablets with placebo tablets. The occurrence of few adverse events among which only one was judged as reasonably attributable to the trial drug proves the excellent safety and tolerance of E₂-CDS when buccally administered at a low dose. The finding of an increase in the urinary 2OHE₁ to 16OHE₁ ratio indicates a good safety profile in terms of breast cancer risk as well. Data from literature has consistently proven that a lower urinary 2OHE₁/16OHE₁ ratio represents an important biomarker for increased breast cancer risk. Treatment with E₂-CDS does not change the metabolism of E₂ and E₁ in a way that would confer an increased risk for breast cancer, but on the contrary changes the ratio in a beneficial direction. The metabolite profile is protective rather than harmful. Because these metabolites compete for the same estrogenic receptor, the increased amount of the “good metabolite” (2OHE₁) decreases the possibility that the “bad metabolite” (16OHE₁) will occupy the estrogen receptor and initiate cellular events that can lead to mutations within breast epithelial cells.

A Phase II clinical trial (first efficacy study or proof-of-concept study) is under preparation. This new clinical study is designed to evaluate primarily the effects of E₂-CDS complexed with HPβCD and delivered by the buccal route (Estredox™), administered once daily (QD) at three dose levels (0.5 mg/day, 1.0 mg/day, and 2.0 mg/day), compared to placebo, during a 12-week treatment phase, on the number and severity of hot flashes as measured by the “hot flash daily weighted severity score” (DWSS) in patients suffering from moderate to severe postmenopausal vasomotor symptoms. Secondary parameters to be evaluated are the placebo-controlled treatment effects on scores calculated from a Menopause Rating Scale (MRS) questionnaire in this patient population. Treatment compliance, and acceptability of the buccal formulation tablet will also be evaluated among the secondary parameters of the study. Disintegration time of the buccal tablets will be recorded on Day 1, 28, and 26. Safety indices before and after treatment will be evaluated too, and include physical examination with vital signs, routine safety laboratory tests, including hemostasis parameters, observed or reported adverse events, hormone levels as biomarkers of central estradiol effects, such as serum FSH, LH, prolactin, SHBG, E₂, E₁, urinary E₁ and the ratio of urinary 2OHE₁ and 16OHE₁, endometrial thickness evaluated by TVS, Pap smear, vaginal cytology (maturation index) and pH, endometrial aspirate with Pipelle, and breast examination.

The primary objective of this study is the evaluation of the effect of QD Estredox™ buccal tablet at doses of 0.5, 1.0, and 2.0 mg E₂-CDS/day compared to placebo on the number and severity of hot flashes in ambulatory postmenopausal women suffering from moderate to severe vasomotor symptoms (hot flashes) during 12 weeks of treatment.

Secondary objectives include the evaluation of placebo-controlled effects of three doses of Estredox™ (0.5, 1.0, and 2.0 mg E₂-CDS/day) on the scores of the MRS questionnaire obtained before, during (at weeks 4 and 8) and after 12 weeks of treatment. Treatment compliance and the acceptability of the buccal tablet are also to be determined and tablet disintegration times are to be recorded on three occasions (Day 1, 28, and 56).

The safety of Estredox™ treatment is to be determined by measuring vital signs, routine laboratory, including hemostasis parameters, and biomarkers to confirm central estrogenic effects, such as serum FSH, LH, E₂ together with prolactin, SHBG, and E₁, urinary E₁ and the ratio of urinary 2OHE₁/16OHE₁ before, during (except prolactin, SHBG, and urine—at weeks 4 and 8) and after the 12 weeks treatment period. Patients are to also undergo detailed gynecological examinations including endometrial thickness by TVS, Pap smear, vaginal cytology (maturation index) and pH, endometrial aspirate with Pipelle, and breast examination (mammography and ultrasound) twice; i.e. before and after treatment (week 0 and 12).

This is to be a phase II multi-center, repeated administration, double-blind, placebo-controlled dose-range study involving 80 ambulatory postmenopausal female patients randomly assigned in equal numbers into one of four treatment groups. Patients with an intact uterus, who are not under current estrogen, or estrogen-progestogen (ET/EPT), phytoestrogen, or selective estrogen receptor modulator (SERM) therapy can be enrolled. If they were under previous ET/EPT, phytoestrogen or SERM therapy, then an appropriate wash-out period will precede the enrollment of potential study candidates into the study. They first will enter a two-week no-treatment run-in phase, during which patients will be required to keep a diary to record the number and severity of hot flashes. Only patients experiencing more than 50 moderate to severe hot flashes per week (>7 per day on average) will be eligible for enrollment into the treatment phase of the study. Eligible patients will be allocated randomly and in equal numbers into one of the four treatment groups. Patients in all treatment groups will receive once daily (QD) in a double-blind fashion either one placebo buccal tablet, or one identical Estredox™ buccal tablet at a dose of 0.5, 1.0, or 2.0 mg E₂CDS/day on each morning of the study, for 84 days, under fasting conditions. The disintegration times of the buccal tablets will be recorded on Days 1, 28, and 56 when patients will self-administer the tablets at the site in the presence of study personnel. During the treatment period, patients will continue the recording of the number and severity of hot flashes. There will be interim assessments of the MRS questionnaire scores after 28 and 56 days of treatment (after weeks 4 and 8) and evaluation of compliance and adverse events. At these interim visits blood will also be sampled for determination of certain hemostasis parameters, and for serum hormone concentrations (E₂, E₁, FSH, LH only). The 12-week treatment period will fully be evaluated on Day 85 during the discharge visit.

Thus, administration of E₂-CDS in accord with the present invention can provide effective treatment of female sexual dysfunction, including effective treatment of postmenopausal symptoms, at doses far lower than previously expected to be effective for treating women with E₂-CDS for postmenopausal symptoms. No specific dosages were ever previously suggested for treating other aspects of female sexual dysfunction such as sexual desire disorders or sexual pain disorders; in fact, treatment of these aspects of female sexual dysfunction has not been previously proposed and no relevant animal testing has been described in the E₂-CDS literature. Moreover, the E₂-CDS literature emphasizes the substantial and prolonged suppression of LH levels. However, while LH inhibition may be more important for certain uses of estrogens such as contraception, there does not appear to be a direct connection between LH suppression and treatment of sexual dysfunction. The low levels of E₂-CDS which can be effectively administered to women for the treatment of various aspects of sexual dysfunction in accord with this invention are particularly surprising; the 0.5 to 2.0 mg daily buccal dose, assuming approximately 30% bioavailability, calculates to an actual useable dose of only 0.15 to 0.6 mg per day, which divided by an average 60-70 kg weight, gives an approximate 0.0025 to 0.01 or less mg/kg dose in women. This is far less than the dose previously expected to be needed to effectively suppress LH and treat postmenopausal symptoms for an extended period. Obviously, dosage amounts will vary with the route of administration and the bioavailability applicable to the selected route. The particular conditions to be relieved by administration in accord with the present invention include female sexual dysfunction, especially of the hypoactive sexual desire disorder type or of the sexual pain disorder type, as well as the symptoms linked to those disorders in postmenopausal women, whether the symptoms are associated with age or with other causes of estrogen deprivation (such as surgery). These include vaginal dryness/lack of lubrication and consequent pain associated with intercourse, vasomotor symptoms such as night sweats and hot flushes, insomnia, depression, nervousness, urinary incontinence, irritability and anxiety, even fear of pain of intercourse, all of which may be associated with the hypoactive sexual desire disorder. Of course, other conditions associated with the estrogen deprivation of menopause or postmenopause, such as osteoporosis and Alzheimer's disease, are also expected to be diminished by administration of the low-dose E₂-CDS formulations provided herein. And these dosages do not provide constant elevated peripheral estrogen levels comparable to pre-menopausal levels, such as produced by standard HRT therapy. Rather, E₂-CDS is believed to be effective in diminishing the symptoms indicated above in amounts which do not elevate average steady-state peripheral estradiol levels to above about 50-60 pg/ml. Indeed, an effective dosage level may be selected in which such average peripheral estradiol levels do not exceed 40 pg/ml, or even 20 pg/ml or lower, with average peak estradiol peripheral levels not above 70-90 pg/ml or even lower. It is important to this invention to use repeated small doses rather than single large ones to produce average peripheral estradiol levels which are low enough (50-60 pg/ml, 40-50 pg/ml, 20 pg/ml or lower, steady-state) and not above an average of about 70-90 pg/ml peak to minimize estrogen exposure in women.

### Investigation of Male Rat Sexual Behavior After Orchidectomy

Rationale

Castration causes the termination of sexual behavior in rats, but the sexual activity of castrated male rats can be reestablished by administration of estradiol. This has also been previously shown for administration of E₂-CDS to castrated male rats in Anderson et al. U.S. Pat. No. 4,863,911. At a single intravenous dose of 3 mg/kg in tests described therein, E₂-CDS was found to improve masculine sexual behavior in rats for 28 days by increasing the pursuit of the female by the male (i.e., decreasing mount and intromission latency) and by increasing initiation of copulatory behavior (increasing mounts and intromission). These data suggested that E₂-CDS is a potent, long-acting stimulant of the proceptive components of masculine sexual behavior. However, estradiol can interfere with ejaculation and the Anderson et al. patent and other publications relating to E₂-CDS do not address the issue of estradiol levels resulting from E₂-CDS administration as to the impact such levels may have on the treatment of all aspects of male sexual dysfunction, including erectile function. Moreover, it is now clear that the drug as used in males in the E₂-CDS literature produces unacceptably high estradiol levels in the serum for extended periods of time.

Circulating luteinizing hormone (LH) is a biomarker reflecting the CNS effects of estradiol. Estrogen diminishes the secretion of luteinizing hormone-releasing hormone (LHRH) and hence reduces the secretion of LH. Therefore, LH and estradiol levels were investigated to measure the central and peripheral effects of E₂-CDS, respectively.

Experimental Design

Adult male Sprague Dawley rats (300-400 g) from Charles River Hungary Ltd., Godollo, Hungary, were used. Animals were kept in community cages (4 animals/cage) in a climate-controlled room (23±2° C.), with a 14 hour light, 10 hour dark cycle of artificial lighting, using reversed light/dark cycle. Female rats weighing 200-250 g were brought to receptivity by subcutaneous injection of estradiol (50 μg/animal) 48 hours before testing and progesterone (0.5 mg/animal) 4 hours prior to the experiments. These hormones were dissolved in sunflower oil.

After establishment of basal behaviors as discussed below, selected animals were orchidectomized via a single midventral incision and were rehoused.

After repeated testing of animals recovered from orchidectomy as discussed below, rats were divided into four groups and treated intravenously, via a single tail vein injection, with one of the following: group 1: control (27% hydroxypropyl-β-cyclodextrin); group 2: 0.03 mg/kg E₂-CDS; group 3: 0.3 mg/kg E₂-CDS; and group 4: 3 mg/kg E₂-CDS.

Mating was observed during the dark cycle in a plexi observation cage in a room where only a dim red light was on. The male was placed in the observation cage 5 minutes prior to the female.

The following parameters were then measured:
Mount latency (ML): the time from the introduction of the female to the initial mount or intromission;
Intromission latency (IL): the time from introduction of the female to the first intromission; and
Ejaculatory latency (EL): the time from the first intromission to ejaculation.

Sessions were considered negative if IL exceeded 15 minutes. EL was only measured to check the result of castration, so as to select only those animals that showed an ejaculation latency greater than 15 minutes.

To establish basal behavior, each male was tested every 5 days until four successive and consistent behavioral patterns were achieved. This pretesting lasted for about four weeks. Approximately half of the animals tested were deemed suitable for orchidectomy.

Twenty-eight days after healing from orchidectomy, the animals were tested again (Day 0) and divided into 4 experimental groups. Only animals displaying ejaculation latencies greater than 15 minutes were included in the study.

Tests of male sexual behavior were conducted 3, 7, 14, 21, 28, 35 and 42 days after drug administration or until the effect disappeared, i.e. until no statistically significant difference was found between groups during two consecutive tests.

Behavioral patterns and related times were recorded manually by skilled observers.

After each testing day, a blood sample was taken from each animal from the retroorbital sinus under light ether anesthesia to determine serum LH and estradiol levels using double antibody and I¹²⁵ isotope-RIA kits, respectively.

Estradiol benzoate and progesterone were obtained from Richter Pharmaceuticals, Ltd., Budapest, Hungary and from Sigma Chemical Co. Inc., Budapest, Hungary, respectively. 2-Hydroxypropyl-β-cyclodextrin was purchased from Cerestar Inc., Hammond, Ind., US. E₂-CDS as a 3% complex with HPβCD (E₂-CDS-CD) was dissolved in distilled water and diluted with 27% HPβCD solution. E₂-CDS-CD was synthesized by Alchem Laboratories Corporation, Alachua, Fla., US.

Behavioral Testing

Four weeks following orchidectomy, groups of rats were treated with one of the following drug doses via tail vein injection: E₂-CDS 0.03, 0.3, and 3 mg/kg. Blood samples were collected by orbital sinus puncture under light ether anesthesia. The samples were stored at 4° C. for one hour and centrifuged at 1,000 g for 10 minutes. Plasma was separated and stored at −80° C. until assayed. Plasma LH concentrations from individual samples were measured by double antibody radioimmunoassay kits obtained from Amersham Pharmacia Biotech, Rome, Italy. Plasma estradiol levels were determined by I¹²⁵ isotope radioimmunoassay kits obtained from BioChem ImmunoSystems. Concentrations of LH and estradiol were calculated by a computerized standard curve program using Prism software (Version 3.0, GraphPad, San Diego, Calif., USA). The limit of detection was 15 pg/ml.

Behavioral changes were analyzed using the Mann-Whitney U test (Siegel, Nonparametric Statistics for the Behavioral Sciences, New York; McGraw-Hill Book Company, Inc., 1956). The Fisher exact test was used for percentage comparisons (Zar, Biostatistical Analysis, Prentice Hall, Inc., Englewood Cliffs, N.J., 1974). Serum LH data were analyzed for each time and treatment group by analysis of variance (ANOVA) followed by Bonferroni posthoc test. Plasma LH and estradiol concentrations were evaluated by the computerized standard curve program of Prism software (Version 3.0, Graph Pad, San Diego, Calif., US).

Results

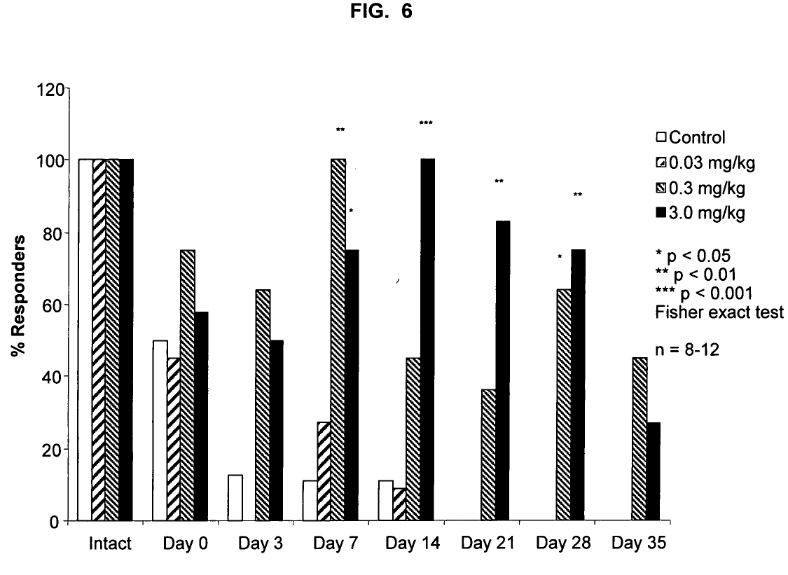

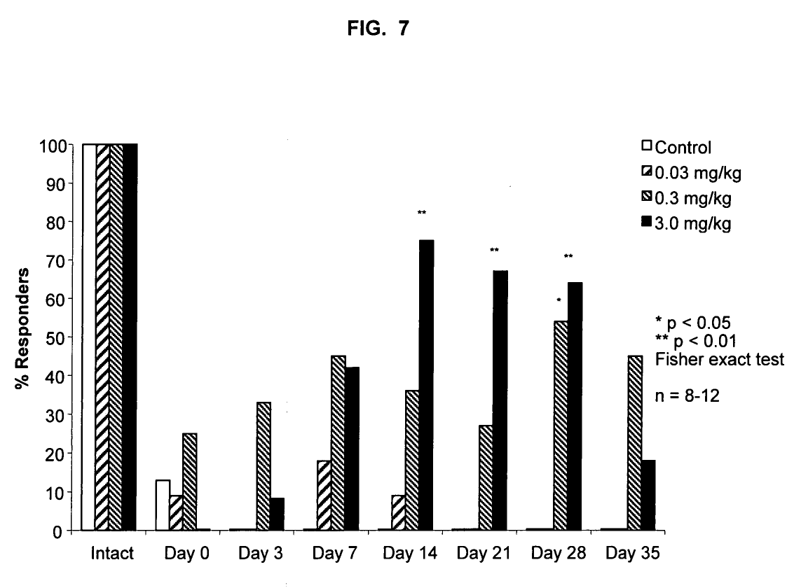

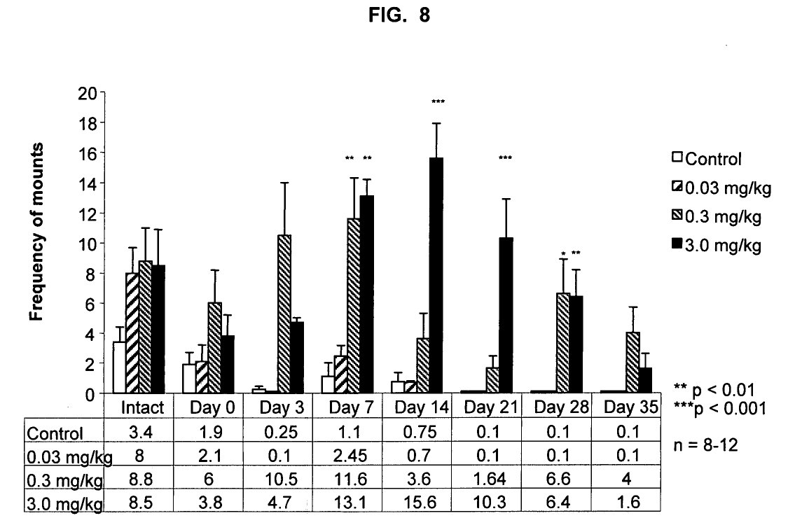

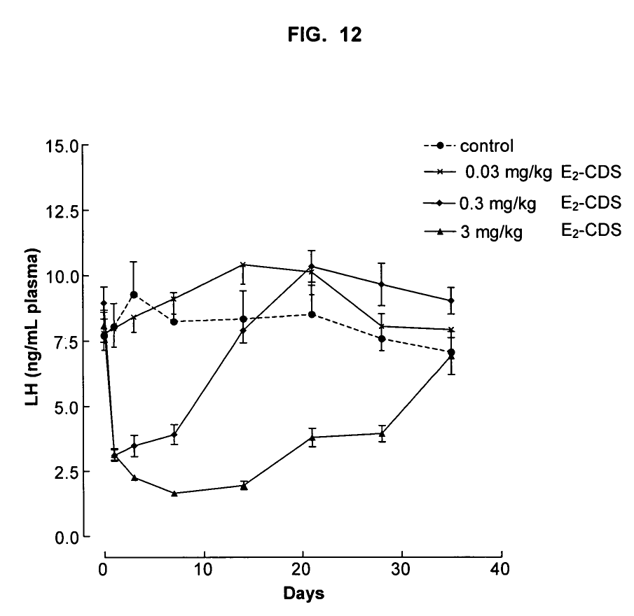

FIGS. 6-12 show the results obtained. In FIGS. 6-12, data are mean±SE for 8-12 animals per group; *p<0.05, **p<0.01, ***p<0.001 using the Fisher exact test or the Mann-Whitney U test, as appropriate (Fisher exact test in FIGS. 6 and 7, Mann-Whitney U tests in FIGS. 8-11). In FIG. 12, each point represents the mean±SEM of samples obtained from 8 to 13 rats.

Orchidectomy was found to be less effective in reducing mounting response (FIG. 6) than in reducing intromission response (FIG. 7). E₂-CDS restored mounting performance in 100% of the animals by day 7 at the dose of 0.3 mg/kg and by day 14 and day 21 at the dose of 3.0 mg/kg. The intromission performance was improved in a statistically significant manner from day 14 through day 28 at the dose of 3.0 mg/kg.

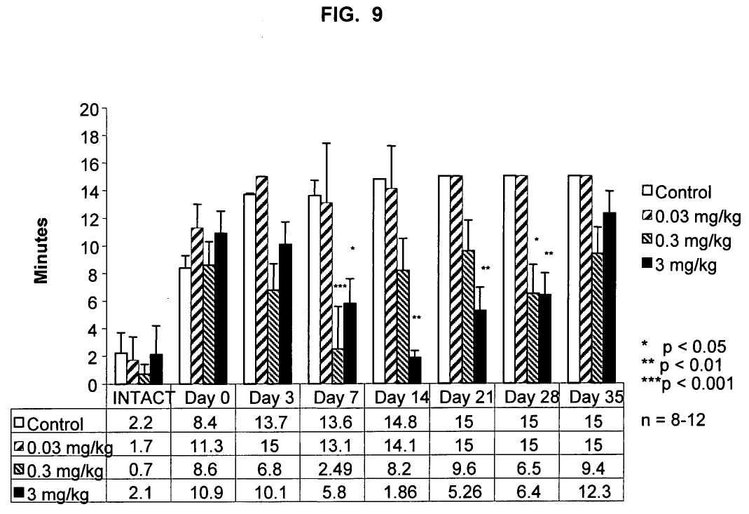

Mount frequency was significantly increased on day 7 at doses of 0.3 and 3.0 mg/kg and on days 14, 21 and 28 at the dose of 3.0 mg/kg (FIG. 8). Mount latency was sharply reduced from day 7 through day 28 for the doses of 0.3 and 3.0 mg/kg (FIG. 9).

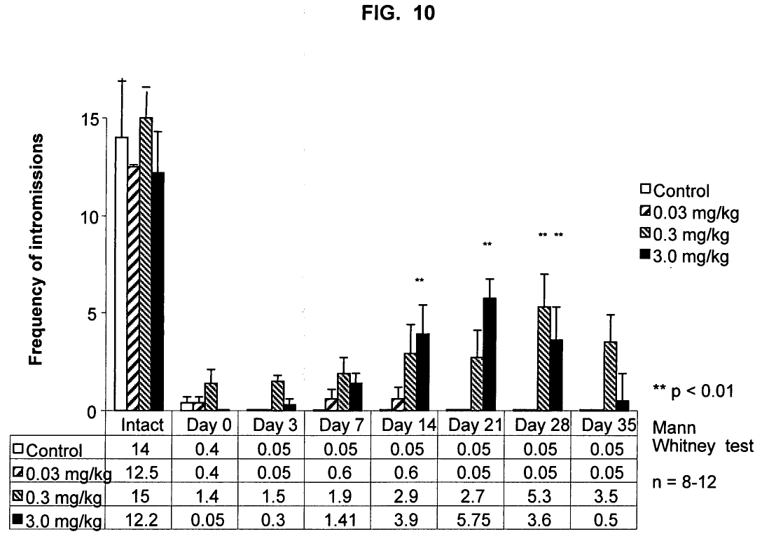

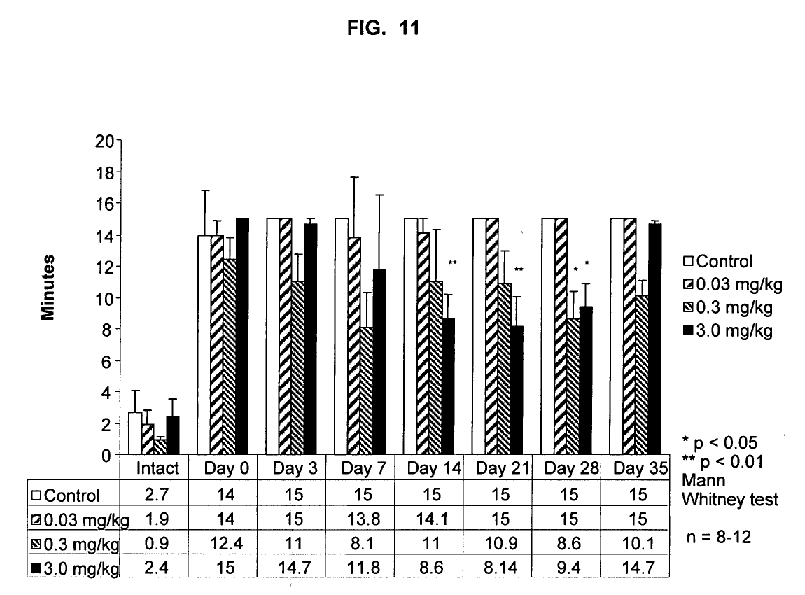

A statistically significant increase in intromission frequency and a decrease in intromission latency was observed on days 14, 21 and 28 at the dose of 3.0 mg/kg (FIGS. 10 and 11).

Thus, the effect of E₂-CDS on the re-establishment of the tested indications of copulatory behavior in male rats was significant at doses of 0.3 and 3.0 mg/kg through day 28. The dose of 0.03 mg/kg had no statistically significant effect.

Concentrations of plasma LH in intact rats were 1.1±0.15 ng/ml. Four weeks after bilateral orchidectomy, LH levels increased to 8.13 ng/ml. At the lowest dose of E₂-CDS tested (0.03 mg/kg i.v.), plasma LH levels were not reduced. At the dose of 0.3 mg/kg i.v., the title compound significantly reduced the LH levels on days 1, 3, and 7. By day 15, there was no significant difference in the LH levels between control and treated animals. At the highest dose of E₂-CDS tested (3 mg/kg i.v.), LH levels were suppressed significantly throughout 28 days (FIG. 12).

Estradiol levels were below the limit of detection in animals treated with E₂-CDS at doses of 0.03 and 0.3 mg/kg i.v. At the highest dose tested (3 mg/kg i.v.), the estradiol level was 258±19 pg/ml on day 1 after treatment. At this dose, the hormone level decreased by 39% to 165±14 pg/ml on day 3 and to 61±7.7 pg/ml on day 7. When next tested on day 14, the estradiol level for the highest dose tested was below the limit of detection. See Table 1 below. This confirms that the dosage level of E₂-CDS used in the Anderson et al. patent (3 mg/kg single i.v. dose in rats) would have produced unacceptably high peripheral estradiol levels for a prolonged period and agrees with data set forth in the Anderson et al. patent and in the E₂-CDS literature. This level is expected to be high enough to interfere with ejaculation.

TABLE 1
Plasma estradiol concentrations following E₂-CDS
or vehicle treatment in orchidectomized rats.

| | Days relative to | Plasma estradiol |
|---|---|---|
| Treatment | treatments | (pg/ml) |
| Vehicle | 0 | ND** (9/9)* |
| | 1 | ND** (9/9)* |
| | 3 | ND** (9/9)* |
| | 7 | ND** (9/9)* |
| | 14 | ND** (9/9)* |
| | 21 | ND** (9/9)* |
| | 28 | ND** (9/9)* |
| | 35 | ND** (9/9)* |
| 0.03 mg/kg E₂-CDS | 0 | 27.3 (9/11)* |
| | 1 | ND** (10/10)* |
| | 3 | ND** (10/10)* |
| | 7 | ND** (10/10)* |
| | 14 | ND** (10/10)* |
| | 21 | ND** (10/10)* |
| | 28 | ND** (10/10)* |
| | 35 | ND** (10/10)* |
| 0.3 mg/kg E₂-CDS | 0 | 31.4 (10/12)* |
| | 1 | ND** (11/11)* |
| | 3 | ND** (11/11)* |
| | 7 | ND** (11/11)* |
| | 14 | ND** (11/11)* |
| | 21 | ND** (11/11)* |
| | 28 | ND** (11/11)* |
| | 35 | ND** (11/11)* |
| 3 mg/kg E₂-CDS | 0 | 54 ± 21 (8/13)* |
| | 1 | 258 ± 19 (0/12)* |
| | 3 | 165 ± 14 (0/12)* |
| | 7 | 61 ± 7.7 (0/12) |
| | 14 | ND** (12/12)* |
| | 21 | ND** (12/12)* |
| | 28 | ND** (12/12)* |
| | 35 | ND** (12/12)* |

*Mean ± SEM; in parentheses: number of samples with estradiol levels below the detection limit (15 pg/ml) of the assay/number of samples per group.
**ND not detectable

The testing described above was repeated using a dose of 0.03 mg/kg administered as a single i.v. injection, and a dose of 0.01 mg/kg with daily i.v. administration for 10 days. E₂-CDS stock solution (40%) was diluted in 27% HPβCD solution.

The copulatory behavior of E₂-CDS treated groups was compared to that of the HPβCD control group at 1, 3, 7, 14, and 21 days after i.v. drug administration in the 0.03 mg/kg group and at 1, 3, 7, 14 and 21 days after initial i.v. drug administration in the 0.01 mg/kg×10 days group.

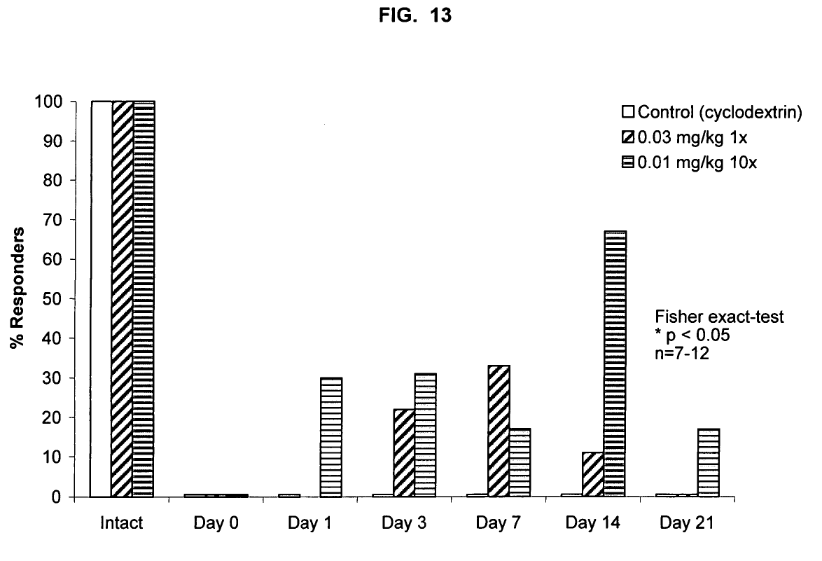

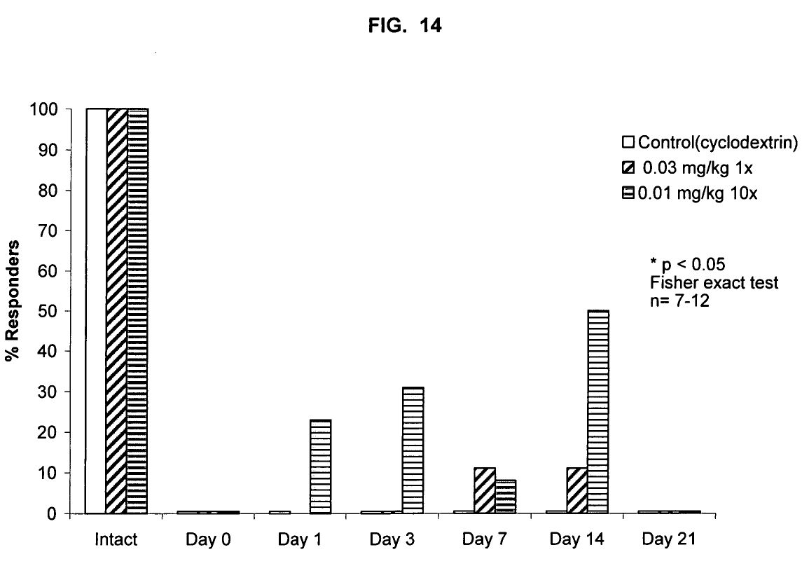

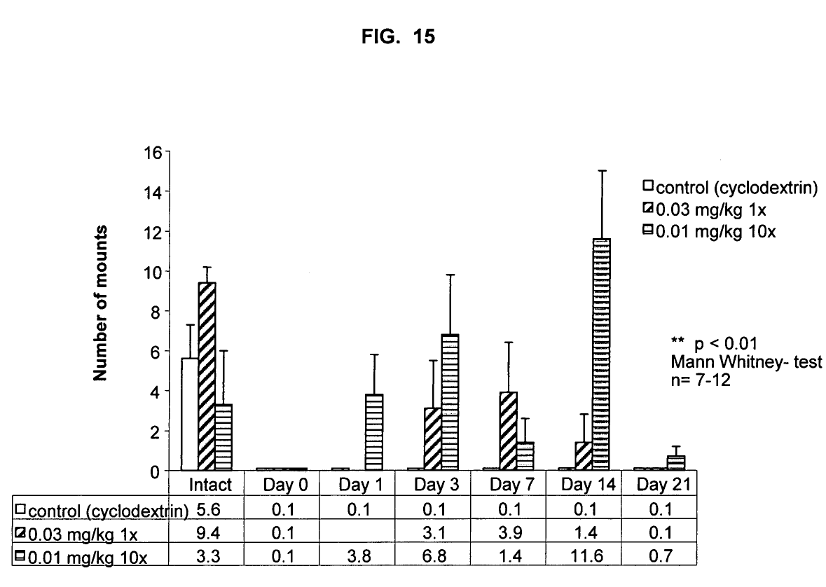

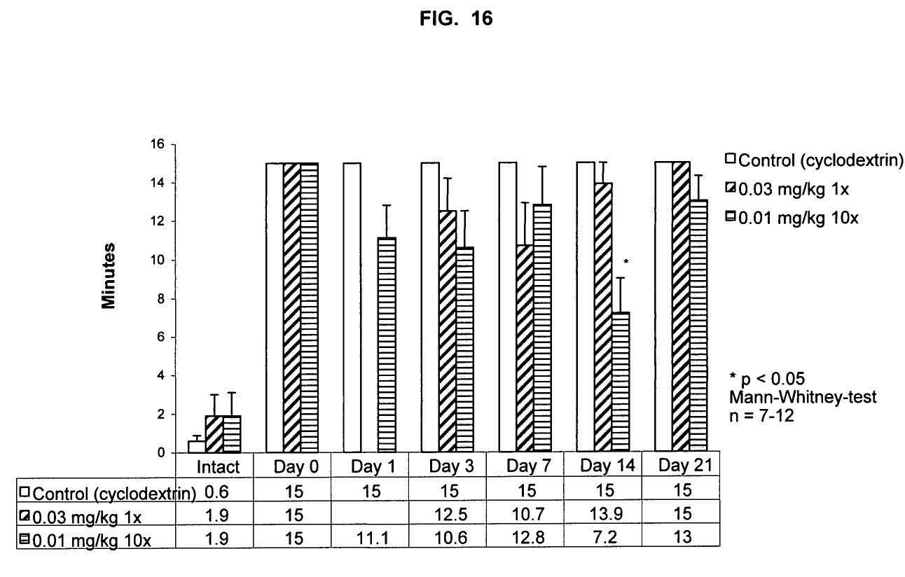

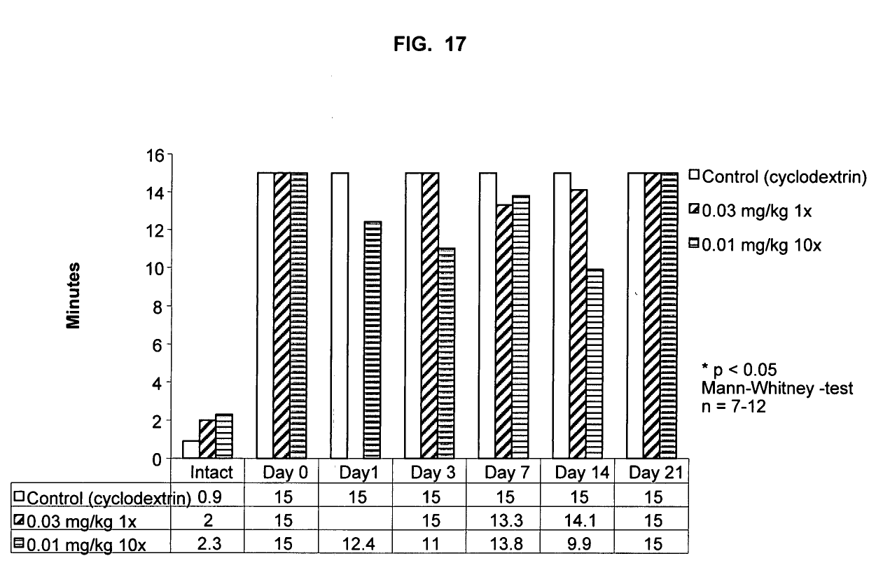

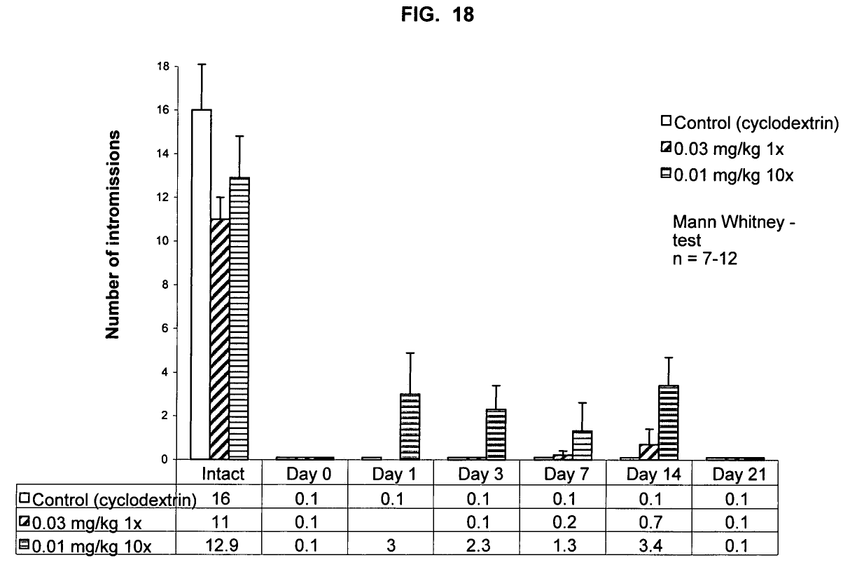

The dose of 0.01 mg/kg administered for 10 days produced significant effect by day 14. It restored mounting performance in 67% and intromission performance in 50% of animals compared to the control group (FIGS. 13 and 14). Mount frequency was increased significantly (FIG. 15). Both mount latency and intromission latency were reduced significantly (FIGS. 16 and 17 ). Intromission frequency was not increased significantly (FIG. 18). One animal, which had good performance before, died on day 7 under the ether anesthesia.

The single dose of 0.03 mg/kg improved sexual activity, but it was not statistically significant in any observations.

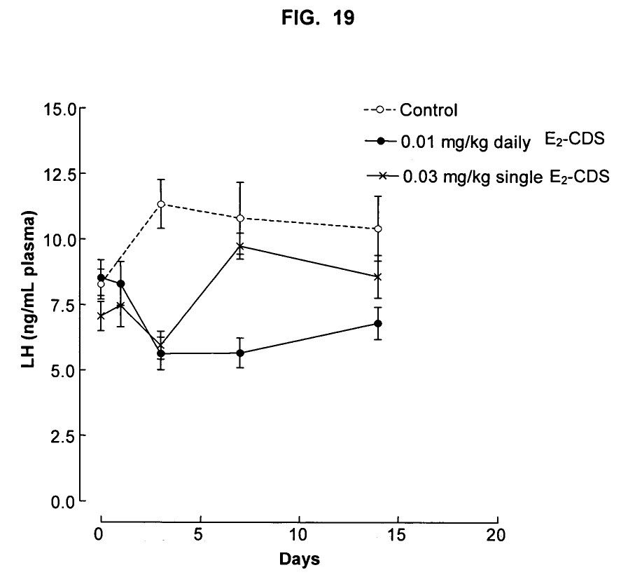

Plasma LH levels were also determined. In the repeated examination, the plasma LH level was significantly reduced at the dose of 0.01 mg/kg (10 daily injections) from day 3 to day 14. At the dose of 0.03 mg/kg (single injection), the plasma LH level was significantly reduced on day 3 only. The results of the repeated examination can be seen in FIG. 19.

At the end of the repeated examination, animals were over-anesthetized, and the prostate and seminal vesicles were removed and their weights were measured. The relative prostate and seminal vesicle weights are summarized in Table 2 below.

TABLE 2
Relative prostate and seminal vesicle weight of
castrated male rats treated with E₂-CDS

| | | Relative seminal |
|---|---|---|
| | Relative^{a} prostate weight | vesicle weight |
| Treatment | Mean ± SE | Mean ± SE |
| HPβCD i.v. | 22.15 ± 2.1 | 25.46 ± 1.55 |
| (control) | | |
| E₂-CDS | 24.78 ± 1.6 | 20.83 ± 0.61** |
| 0.01 mg/kg 10x i.v. | | |
| E₂-CDS | 14.48 ± 2.1* | 19.18 ± 0.82** |
| 0.03 mg/kg 1x i.v. | | |

^{a}mg/100 g body weight; n = 7-12
*p < 0.05,
**p < 0.01 Student's t test compared to control

Estradiol levels were below the limit of detection in all animals treated with E₂-CDS at doses of 0.03 mg/kg (single dose) and 0.01 mg/kg (daily for 10 days) i.v. See Table 3 below.

TABLE 3
Plasma estradiol concentrations following E₂-CDS or
vehicle treatment in orchidectomized rats.

| | Days relative to | Plasma estradiol |
|---|---|---|
| Treatment | treatments | (pg/ml) |
| Vehicle | 0 | ND** (7/7)* |
| | 1 | ND** (7/7)* |
| | 3 | ND** (7/7)* |
| | 7 | ND** (7/7)* |
| | 14 | ND** (7/7)* |
| | 21 | ND** (7/7)* |
| 0.01 mg/kg E₂-CDS | 0 | ND** (12/12)* |
| daily for 10 days i.v. | 1 | ND** (12/12)* |
| | 3 | ND** (12/12)* |
| | 7 | ND** (12/12)* |
| | 14 | ND** (12/12)* |
| | 21 | ND** (12/12)* |
| 0.03 mg/kg E₂-CDS | 0 | ND** (9/9)* |
| single dose i.v. | 1 | ND** (9/9)* |
| | 3 | ND** (9/9)* |
| | 7 | ND** (9/9)* |
| | 14 | ND** (9/9)* |
| | 21 | ND** (9/9)* |

*Mean ± SEM; in parentheses: number of samples with estradiol levels below the detection limit (15 pg/ml) of the assay/number of samples per group.
**ND not detectable.

The foregoing studies show that E₂-CDS can restore male sexual function in rats and indicate that symptoms of male sexual dysfunction in males, including men, can be alleviated through it administration at doses far lower than previously thought possible, while maintaining appropriate peripheral levels of estrogen. Clinical studies in women substantiate that low dose buccal administration of E₂-CDS can be correlated with animal test data and allow calculation of suitable buccal dosages for men based on the animal test data in male rats.

Administration of E₂-CDS in accord with the present invention provides effective treatment of male sexual dysfunction, at doses far lower than previously expected to be effective for treating men with E₂-CDS for male sexual dysfunction by using repeated small doses of the compound rather than the single dose once-a-month therapy suggested earlier, to minimize or obviate elevation of peripheral estradiol levels. It also is not necessary to use a dosage high enough to significantly reduce serum LH in order to effectively treat male sexual dysfunction. The low levels of E₂-CDS which can be effectively administered to men for these purposes are particularly surprising; for example, a dose comparable to 0.01 to 0.001 mg/kg i.v. in the male rat, or a 0.01 to 0.5 mg daily buccal dose in men, is contemplated; assuming approximately 30% bioavailability, this buccal dose calculates to an actual useable dose of only 0.003 to 0.015 mg per day, which divided by an average 70-80 kg weight, gives an approximate 0.0000375 to 0.00021 or less mg/kg dose in men. Treatment is continued once-a-day or once every other day for such period of time as required until symptoms diminish, generally about 2 to 7 days in men, and treatment is resumed when symptoms recur. Obviously, dosage amounts will vary with the route of administration and the bioavailability applicable to the selected route. In any event, the method of administering E₂-CDS in accord with the present invention will utilize dosage amounts and dosage frequencies which will not substantially elevate average peripheral estradiol levels to above average normal levels in the male, i.e., will not elevate average peripheral estradiol levels more than about 10-15% above normal levels. This in turn will prevent peripheral estradiol levels from inhibiting ejaculation, so that both proceptive and consummatory aspects of male sexual behavior will be improved.

For use herein, E₂-CDS can be administered by a variety of routes of administration and dosage forms which are already known from the various Bodor, Bodor et al. and Anderson et al. patents referenced hereinabove, all of which are incorporated by reference herein in their entireties and relied upon. This is also apparent from the dosage forms employed in the animal and human testing described hereinabove.

Pharmaceutical formulations for use in the methods of this invention comprise an amount of E₂-CDS sufficient to diminish symptoms of female sexual dysfunction, including postmenopausal symptoms, which does not elevate average steady-state peripheral estradiol levels above about 50-60 pg/ml, or an amount of E₂-CDS sufficient to diminish symptoms of male sexual dysfunction, which does not substantially elevate average peripheral levels above average normal peripheral levels in the male mammal. The carrier and any other ingredients must of course be compatible with E₂-CDS, and not detrimentally affect the effectiveness of the compound in treating female or male sexual dysfunction.

Suitable pharmaceutically acceptable carriers for use with E₂-CDS are non-toxic and will be apparent to those skilled in the art of pharmaceutical formulation. See, e.g., Remington's Pharmaceutical Sciences, 17^{th} Ed., Gennaro, ed., Mack Publishing Company, Easton, Pa. (1985). The choice of suitable carriers will depend upon the exact nature of the particular dosage form selected, including of course the route of administration. The therapeutic dosage ranges for administration of E₂-CDS for use in treating male and female sexual dysfunction has been detailed hereinabove. The dose selected will of course vary with the severity of the symptoms treated, the route of administration, the dosage form and the like. Formulations according to the invention may be administered in any therapeutically effective manner, including, but not limited to, bucally, intranasally, sublingually, orally, topically (dermally), parenterally, by inhalation spray, vaginally or rectally in dosage unit formulations.

Thus, for example, the pharmaceutical compositions according to the invention may be administered parenterally. The term “parenterally” as used herein includes, but is not limited, to subcutaneous injections, intravenous, intramuscular, intrastemal injection or infusion techniques. The pharmaceutical composition may be in the form of a sterile injectable aqueous or oleagenous suspension. This suspension may be formulated according to known methods using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. The acceptable vehicles and solvents include but are not limited to freshly prepared aqueous cyclodextrin solutions, particularly of hydroxyalkyl derivatives of β- or γ-cyclodextrin or carboxyalkyl derivatives of β- or γ-cyclodextrin or carboxymethylethyl β- or γ-cyclodextrin or other suitable derivative, as discussed in more detail below. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Alternatively, and most desirably for human use, the pharmaceutical compositions according to the invention may be administered through buccal drug delivery. The term “buccal” refers to delivery of a drug by passage of a drug through the buccal mucosa into the bloodstream. Buccal drug delivery can be effected by placing the buccal dosage unit between the lower gum and the oral mucosa opposite thereto of the individual undergoing drug therapy. Excipients or vehicles suitable for buccal drug administration can be used, and include any such materials known in the art, e.g., any liquid, gel, solvent, liquid diluent, solubilizer, or the like, which is nontoxic and does not interact with other components of the composition in a deleterious manner. The dosage unit is fabricated so as to dissolve gradually over a predetermined time period, to produce a substantially saturated drug solution in the saliva of the buccal cavity, allowing absorption of E₂-CDS through the mucosa, wherein drug delivery is provided essentially throughout the time period. The buccal dosage unit may further comprise a lubricant to facilitate manufacture, e.g., magnesium stearate or the like. Additional components that may be included in the buccal dosage unit include but are not limited to flavorings, permeation enhancers, diluents, binders, and the like. The remainder of the buccal dosage unit comprises the bioerodible polymeric carrier, and any excipients that may be desired, e.g., binders, disintegrants, lubricants, diluents, flavorings, colorings, and the like, and/or additional active agents.

The buccal carrier can comprise a polymer having sufficient tack to ensure that the dosage unit adheres to the buccal mucosa for the necessary time period, i.e., the time period during which the E₂-CDS is to be delivered to the buccal mucosa. Additionally, the polymeric carrier is gradually “bioerodible”, i.e., the polymer hydrolyzes at a predetermined rate upon contact with moisture. Any polymeric carriers can be used that are pharmaceutically acceptable, provide both a suitable degree of adhesion and the desired drug release profile, and are compatible with the E₂-CDS to be administered and any other components that may be present in the buccal dosage unit. Generally, the polymeric carriers comprise hydrophilic (water-soluble and water-swellable) polymers that adhere to the wet surface of the buccal mucosa. Examples of polymeric carriers useful herein include acrylic acid polymers and copolymers, e.g., those known as “carbomers” for example, Carbopol®. Other suitable polymers include, but are not limited to hydrolyzed polyvinyl alcohol, polyethylene oxides (e.g., Sentry Polyox®), polyacrylates (e.g., Gantrez®), vinyl polymers and copolymers, polyvinylpyrrolidone; dextran, guar gum, pectins, starches, and cellulosic polymers such as hydroxypropyl methylcellulose (e.g., Methocel®), hydroxypropyl cellulose (e.g., Klucel®), hydroxypropyl cellulose ethers, hydroxyethyl cellulose, sodium carboxymethyl cellulose, methyl cellulose, ethyl cellulose, cellulose acetate phthalate, cellulose acetate butyrate, and the like. The dosage unit need contain only the E₂-CDS and the carrier. However, it may be desirable in some cases to include one or more additional components. For example, a lubricant may be included to facilitate the process of manufacturing the dosage units; lubricants may also optimize erosion rate and drug flux. If a lubricant is present, it will represent on the order of 0.01 wt. % to about 2 wt. %, preferably about 0.01 wt. % to 0.5 wt. %, of the dosage unit. Suitable lubricants include, but are not limited to, magnesium stearate, calcium stearate, stearic acid, sodium stearylfumarate, talc, hydrogenated vegetable oils and polyethylene glycol. In any event, the E₂-CDS will most desirably be incorporated into the buccal dosage form as a complex, preferably a substantially saturated complex, with a hydroxyalkyl or carboxyalkyl or carboxymethylethyl or other derivative of β- or γ-cyclodextrin, as discussed in more detail hereinbelow.

E₂-CDS may also be administered in accord with this invention in the form of a transdermal patch for transdermal administration of the drug. The transdermal patches may include a variety of additional excipients which are conventionally employed to facilitate the transdermal administration of an active agent. Examples of such excipients include but are not limited to carriers, gelling agents, suspending agents, penetration-enhancing agents, dispersing agents, preservatives, stabilizers, wetting agents, emulsifying agents, and the like. Specific examples of each of these types of excipients are well-known in the art and any conventional excipients may be employed in the transdermal patches. Examples of suitable permeable surface layer materials are also well-known in the art of transdermal patch delivery, and any conventional material which is permeable to the E₂-CDS to be administered. Specific examples of suitable materials for the permeable surface layer include but are not limited to dense or microporous polymer films such as those comprised of polycarbonates, polyvinyl chlorides, polyamides, modacrylic copolymers, polysulfones, halogenated polymers, polychloroethers, acetal polymers, acrylic resins, and the like. Specific examples of these types of conventional permeable membrane materials are described in U.S. Pat. No. 3,797,494. Additionally, suitable penetration-enhancing agents are well known in the art as well. Examples of conventional penetration-enhancing agents include alkanols such as ethanol, hexanol, cyclohexanol, and the like; hydrocarbons such as hexane, cyclohexane, isopropylbenzene, aldehydes and ketones such as cyclohexanone, acetamide, N,N-di(lower alkyl)acetamides such as N,N-diethylacetamide, N,N-dimethylacetamide, N-(2-hydroxyethyl)acetamide, esters such as N,N-di-(lower alkyl)sulfoxides, essential oils such as propylene glycol, glycerine, glycerol monolaurate, isopropyl myristate, and ethyl oleate, salicylates, and mixtures of any of the above. Again, as discussed in more detail hereinbelow, E₂-CDS is advantageously used in transdermal formulations as a complex, especially a substantially saturated complex, with a hydroxyalkyl or carboxyalkyl or carboxymethylethyl derivative of β- or γ-cyclodextrin.

E₂-CDS may also be administered in accord with this invention in the form of suppositories for vaginal or rectal administration. These compositions can be prepared by mixing E₂-CDS (advantageously as a complex, especially a saturated complex, with hydroxypropyl-β-cyclodextrin, hydroxyethyl-β-cyclodextrin, the corresponding hydroxyethyl-β- or γ-cyclodextrin derivative or the carboxymethyl or carboxyethyl or carboxymethylethyl derivative of β- or γ-cyclodextrin) with a suitable non-irritating excipient or binder which is solid at ordinary temperatures but liquid at the vaginal or rectal temperature and will, therefore, melt in the vagina or rectum to release the drug. Such materials are cocoa butter and polyethylene glycols. Traditional binders and carriers include, for example, polyalkylene glycols or triglycerides [e.g., PEG 1000 (96%) and PEG 4000 (4%)]. Such suppositories may be formed from mixtures containing active ingredients in the range of from about 0.5 wt/wt % to about 10 wt/wt %; preferably from about 1 wt/wt % to about 2 wt/wt %.

For topical/dermal use, creams, ointments, jellies, solutions or suspensions, etc., containing E₂-CDS (desirably complexed with one of the cyclodextrin derivatives named in the preceding paragraph) can be employed.

For intranasal use, a powder spray, suspension, gel or ointment may be utilized, preferably a powder form of E₂-CDS, which may be complexed with one of the cyclodextrin derivatives discussed in the preceding two paragraphs.

It will be apparent to those familiar with the patent and non-patent literature regarding E₂-CDS, that complexation with cyclodextrin derivatives as well as formulation with such derivatives provides particularly useful dosage forms of E₂-CDS for a variety of routes of administration. See, for example Bodor U.S. Pat. Nos. 5,017,566; 5,002,935; 4,983,586; and 5,024,998; all of which are incorporated by reference herein in their entireties and relied upon. See also patents describing hydroxyalkylated derivatives of β- and γ-cyclodextrin such as Pitha U.S. Pat. Nos. 4,596,795 and 4,727,064 and Müller U.S. Pat. Nos. 4,764,604 and 4,870,060 and Müller et al. U.S. Pat. No. 6,407,079.

Cyclodextrins of particular interest for complexation with E₂-CDS include: hydroxyalkyl, e.g. hydroxyethyl or hydroxypropyl, derivatives of β- and γ-cyclodextrin; carboxyalkyl, e.g. carboxymethyl or carboxyethyl, derivatives of β- or γ-cyclodextrin; β-cyclodextrin sulfobutyl ether; carboxymethylethyl-β- or γ-cyclodextrin; dimethyl-β-cyclodextrin; and randomly methylated β-cyclodextrin. 2-Hydroxypropyl-β-cyclodextrin (HPβCD), 2-hydroxypropyl-γ-cyclodextrin (HPγCD), randomly methylated β-cyclodextrin, dimethyl-β-cyclodextrin, β-cyclodextrin sulfobutyl ether, carboxymethyl-β-cyclodextrin (CMβCD), carboxymethyl-γ-cyclodextrin (CMγCD) and carboxymethylethyl-β-cyclodextrin are of special interest, especially hydroxypropyl-β-cyclodextrin, hydroxypropyl-γ-cyclodextrin, carboxymethyl-β-cyclodextrin and carboxymethyl-γ-cyclodextrin. 2-Hydroxypropyl-β-cyclodextrin (HPβCD) and 2-hydroxypropyl-γ-cyclodextrin (HPγCD) as well as carboxymethyl-β-cyclodextrin (CMβCD) and carboxymethyl-γ-cyclodextrin (CMγCD), are of special value for complexation, and incorporation of E₂-CDS into pharmaceutical formulations as a HPβCD-complex or HPγCD-complex or CMβCD-complex or CMγCD-complex is highly desirable. Moreover, for use in men or women, a buccal dosage form, especially a buccal tablet or wafer or disk, advantageously having a disintegration time of about 15-30 minutes, or a buccal patch (in which the drug is released only from the side which adheres to the buccal mucosa while the other side is nonpermeable), has particular advantages as it can be readily self-administered yet provides better bioavailability than oral dosage forms because the E₂-CDS passes directly into the bloodstream from the buccal mucosa. (The cyclodextrin derivative is not absorbed, of course.) The formulations for buccal administration are preferably anhydrous for reasons of storage stability. In lower animals, parenteral dosage forms are often considered more practical and of course provide better bioavailability. HPβCD is also advantageously used in parenteral dosage forms for E₂-CDS, both as a complexing agent and as a solvent as seen in the animal tests described hereinabove. HPγCD, CMβCD or CMγCD, hydroxyethyl-β-cyclodextrin, hydroxyethyl-γ-cyclodextrin, carboxyethyl-β-cyclodextrin or carboxyethyl-γ-cyclodextrin may, for example, be used instead.

In particularly advantageous embodiments of the invention, buccal administration may make use of the inventions of Nagai et al described in U.S. Pat. Nos. 4,226,848 and 4,250,163, both of which are incorporated by reference herein in their entireties and relied upon. Thus, a buccal mucosa-adhesive tablet may be formulated for use herein comprising: (a) a water-swellable and mucosa-adhesive polymeric matrix comprising about 50% to about 95% by weight of a cellulose ether and about 50% to about 95% by weight of a homo- or copolymer of acrylic acid or a pharmaceutically acceptable salt thereof, and (b) dispersed therein, an appropriate quantity of E₂-CDS, typically from about 0.5 to 2.0 mg for use in women and a lesser amount for use in men, as a substantially saturated complex with 2-hydroxypropyl-β-cyclodextrin. Ideally, for storage stability, the tablet is anhydrous. Again, one of the aforementioned other cyclodextrins, e.g. hydroxyalkyl or carboxyalkyl or carboxymethylethyl β- or γ-cyclodextrin derivatives, may be utilized in place of the 2-hydroxypropyl-β-cyclodextrin.

While the buccal compositions according to the invention may optionally include one or more excipients or other pharmaceutically inert components, one of the advantages of these dosage forms when they comprise E₂-CDS as a cyclodextrin complex is that they can be prepared with the minimal amount of excipients necessary for shaping and producing the particular form, such as a tablet or patch. Excipients may be chosen from those that do not interfere with the E₂-CDS, with cyclodextrin or with complex formation. A simple solid buccal dosage form consists of the substantially saturated E₂-CDS-cyclodextrin complex compressed with a small amount (e.g. about 1% by weight) of a suitable binder or lubricant such as magnesium stearate. Sorbitol may be added to the complex as well as magnesium stearate to aid in fast dissolution and to give good mouth feel.

The buccal dosage form may be a liquid. In that case, it can for example be obtained by dissolving a substantially saturated complex of E₂-CDS in cyclodextrin in a minimum amount of water, for example 500 mg of the substantially saturated complex with HPβCD in 0.5 mL water (50% w/w solution), or 500 mg of the substantially saturated γCD complex in 1.0 mL of water. A few drops of such a solution can be inserted into the buccal cavity and retained there for about 2 minutes to allow for absorption through the buccal mucosa. Nevertheless, solid buccal or other transmucosal dosage forms are generally preferred over liquid forms.

As will be apparent to those skilled in the art to which the invention pertains, the present invention may be embodied in forms other than those specifically disclosed above without departing from the spirit or essential characteristics of the invention. The particular embodiments of the invention described above are, therefore, to be considered as illustrative and not restrictive. The scope of the invention is as set forth in the appended claims rather than being limited to the foregoing description.

1. A method for the treatment of female sexual dysfunction in a female mammal in need of such treatment, said method comprising administering to said mammal the compound 17β-[(1-methyl-1,4-dihydro-3-pyridinyl)carbonyloxy]estra-1,3,5(10)-trien-3-ol, in an amount effective to diminish symptoms of said dysfunction which does not elevate average steady-state peripheral estradiol levels to above about 50-60 pg/ml.

2. A method according to claim 1, wherein the amount administered is equivalent in bioavailability to a dose of about 0.03 mg/kg or less per day when administered intravenously to ovariectomized female rats.

3. A method according to claim 1, wherein the amount administered does not elevate average steady-state peripheral estradiol levels to above about 40 pg/ml.

4. A method according to claim 3, wherein the amount administered does not elevate average steady-state peripheral estradiol levels to above about 20 pg/ml or lower.

5. A method according to claim 1, wherein the amount administered does not provide average peak peripheral estradiol levels above about 70-90 pg/ml or lower.

6. A method according to Claim, wherein the female mammal is a woman.

7. A method according to claim 6, wherein the amount administered is about 0.01 mg/kg or less per day, and is administered buccally.

8. A method according to claim 7, wherein the amount administered is from about 0.5 to about 2.0 mg/day.

9. A method according to claim 6, wherein the amount administered does not elevate average steady-state peripheral estradiol levels to above about 40 pg/ml.

10. A method according to claim 9, wherein the amount administered does not elevate average steady-state peripheral estradiol levels to above about 20 pg/ml or lower.

11. A method according to claim 6, wherein the amount administered does not provide average peak peripheral estradiol levels above about 70-90 pg/ml or lower.

12. A method according to claim 6, wherein the female sexual dysfunction comprises hypoactive sexual desire type female sexual dysfunction and/or sexual pain type female sexual dysfunction.

13. A method according to claim 6, wherein the female sexual dysfunction is accompanied by postmenopausal-type symptoms including at least one member selected from the group consisting of vaginal dryness/lack of lubrication, night sweats, hot flushes, insomnia, depression, nervousness, urinary incontinence, irritability and anxiety.

14. A method according to claim 1, wherein the compound is administered as a substantially saturated complex with: a hydroxyalkyl or carboxyalkyl derivative of β- or γ-cyclodextrin; carboxymethylethyl-β- or -γ-cyclodextrin; β-cyclodextrin sulfobutyl ether; dimethyl-β-cyclodextrin; or randomly methylated β-cyclodextrin.

15. A method according to claim 14, wherein the compound is administered as a substantially saturated complex with hydroxypropyl-β-cyclodextrin, hydroxypropyl-γ-cyclodextrin, hydroxyethyl-β-cyclodextrin, hydroxyethyl-γ-cyclodextrin, carboxymethyl-β-cyclodextrin, carboxymethyl-γ-cyclodextrin, carboxyethyl-β-cyclodextrin or carboxyethyl-γ-cyclodextrin.

16. A method according to claim 15, wherein the compound is administered as a substantially saturated complex with 2-hydroxypropyl-β-cyclodextrin or 2-hydroxypropyl-γ-cyclodextrin.

17. A method according to claim 14, wherein the complex is administered in an anhydrous formulation.

18. A method according to claim 17, wherein the anhydrous formulation is a buccal tablet, buccal wafer or buccal patch.

19. A method for the treatment of postmenopausal symptoms in a postmenopausal woman in need of same, said method comprising administering to said woman the compound 17β-[(1-methyl-1,4-dihydro-3-pyridinyl)carbonyloxy]estra-1,3,5(10)-trien-3-ol, in an amount effective to diminish said symptoms which does not elevate average steady-state peripheral estradiol levels to above about 50-60 pg/ml.

20. A method according to claim 19, wherein the amount administered is about 0.01 mg/kg or less per day, and is administered buccally.

21. A method according to claim 20, wherein the amount administered is from about 0.5 to about 2.0 mg/day.

22. A method according to claim 19, wherein the amount administered does not elevate average steady-state peripheral estradiol levels to above about 40 pg/ml.

23. A method according to claim 22, wherein the amount administered does not elevate average steady-state peripheral estradiol levels to above about 20 pg/ml or lower.

24. A method according to claim 19, wherein the amount administered does not provide average peak peripheral estradiol levels to above about 70-90 pg/ml or lower.

25. A method according to claim 19, wherein said postmenopausal symptoms are associated with female sexual dysfunction.

26. A method according to claim 25, wherein said female sexual dysfunction is of the hypoactive sexual desire type or of the sexual pain type, or both.

27. A method according to claim 19, wherein the postmenopausal symptoms include at least one member selected from the group consisting of vaginal dryness/lack of lubrication, night sweats, hot flushes, insomnia, depression, nervousness, urinary incontinence, irritability and anxiety.

28. A method according to claim 19, wherein the compound is administered as a substantially saturated complex with: a hydroxyalkyl or carboxyalkyl derivative of β- or γ-cyclodextrin; carboxymethylethyl-β- or γ-cyclodextrin; β-cyclodextrin sulfobutyl ether; dimethyl-β-cyclodextrin; or randomly methylated β-cyclodextrin.

29. A method according to claim 28, wherein the compound is administered as a substantially saturated complex with hydroxypropyl-β-cyclodextrin, hydroxypropyl-γ-cyclodextrin, hydroxyethyl-β-cyclodextrin, hydroxyethyl-γ-cyclodextrin, carboxymethyl-β-cyclodextrin, carboxyethyl-β-cyclodextrin, carboxymethyl-γ-cyclodextrin or carboxyethyl-γ-cyclodextrin.

30. A method according to claim 29, wherein the compound is administered as a substantially saturated complex with 2-hydroxypropyl-β-cyclodextrin or 2-hydroxypropyl-γ-cyclodextrin.

31. A method according to claim 28, wherein the complex is administered in an anhydrous formulation.

32. A method according to claim 31, wherein the anhydrous formulation is a buccal tablet, buccal wafer or buccal patch.

33. A method for the treatment of male sexual dysfunction in a male mammal in need of such treatment, said method comprising administering to said mammal the compound 17β-[(1-methyl-1,4-dihydro-3-pyridinyl)carbonyloxy]estra-1,3,5(10)-trien-3-ol, in an amount effective to diminish symptoms of said dysfunction which does not substantially elevate average peripheral estradiol levels to above average normal peripheral estradiol levels in the male mammal.

34. A method according to claim 33, wherein the amount administered is equivalent in bioavailability to a dose of from about 0.01 to about 0.001 mg/kg per day when administered intravenously to castrated male rats.

35. A method according to claim 33, wherein the amount is administered once a day or once every other day until said symptoms diminish.

36. A method according to claim 35, wherein treatment is resumed when symptoms recur.

37. A method according to claim 33, wherein the male mammal is a man.

38. A method according to claim 37, wherein the amount administered is from about 0.01 to about 0.5 mg/day, and is administered bucally.

39. A method according to claim 37, wherein the amount is administered once a day or once every other day until said symptoms diminish.

40. A method according to claim 39, wherein the treatment period is for about 2 to 7 days.

41. A method according to claim 40, wherein treatment is resumed when symptoms recur.

42. A method according to claim 33, wherein the compound is administered as a substantially saturated complex with: a hydroxyalkyl or carboxyalkyl derivative of β- or γ-cyclodextrin; carboxymethylethyl-β- or γ-cyclodextrin; β-cyclodextrin sulfobutyl ether; dimethyl-β-cyclodextrin; or randomly methylated β-cyclodextrin.

43. A method according to claim 42, wherein the compound is administered as a substantially saturated complex with hydroxypropyl-β-cyclodextrin, hydroxypropyl-γ-cyclodextrin, hydroxyethyl-β-cyclodextrin, hydroxyethyl-γ-cyclodextrin, carboxymethyl-β-cyclodextrin, carboxymethyl-γ-cyclodextrin, carboxyethyl-β-cyclodextin or carboxyethyl-γ-cyclodextrin.

44. A method according to claim 43, wherein the compound is administered as a substantially saturated complex with 2-hydroxypropyl-β-cyclodextrin or 2-hydroxypropyl-γ-cyclodextrin.

45. A method according to claim 42, wherein the complex is administered in an anhydrous formulation.

46. A method according to claim 45, wherein the anhydrous formulation is a buccal tablet, buccal wafer or buccal patch.
